(19) United States
(12) Patent Application Publication
(10) Pub. No.: US 2026/0308810 A1
(43) Pub. Date: Oct. 8, 2026

(54) Method, Apparatus and Composition for Fibre Deposition

(71) Applicant: CAMBRIDGE ENTERPRISE LIMITED, Cambridge, Cambridgeshire (GB)

(72) Inventors: Yan Yan Shery Huang, Cambridge, Cambridgeshire (GB); Wenyu Wang, Cambridge, Cambridgeshire (GB); Yifei Pan, Cambridge, Cambridgeshire (GB); Yang Cao, Cambridge, Cambridgeshire (GB)

(21) Appl. No.: 19/480,570

(22) PCT Filed: May 3, 2024
(86) PCT No.: PCT/GB2024/051179

§ 371 Date: Oct. 31, 2025

(30) Foreign Application Priority Data
May 18, 2023 (GB) 2307449.5

(51) Int. Cl. D01D 5/04; A61B 5/27; D01D 1/02; D01D 1/06; D04H 1/736; D04H 1/74; D04H 1/76

(52) U.S. Cl. CPC D01D 5/04; D01D 1/02; D01D 1/065; D04H 1/736; D04H 1/74; D04H 1/76; A61B 5/27; D10B 2321/121; D10B 2331/06; D10B 2331/30; D10B 2403/0243

(57) ABSTRACT

According to the present disclosure there is provided a method of depositing a fibre onto a freestanding object using a fibre deposition apparatus. The fibre deposition apparatus comprises a dispenser and a moveable arm defining a deposition zone. The method comprises: positioning the fibre deposition apparatus such that the freestanding object is located within the deposition zone, forming a droplet of a fibre-forming liquid composition at an outlet of the dispenser, moving the arm from a first position in contact with the droplet to a second position away from the first position so as to draw the droplet into a drawn fibre, and depositing the drawn fibre onto the freestanding object as the arm moves from the first position to the second position. According to a further aspect of the present disclosure there is provided a fibre deposition apparatus for depositing fibres onto a freestanding object. According to a further aspect of the present disclosure there is provided a liquid composition for forming a drawn fibre. The liquid composition comprises a fibre-forming polymer; a solvent; and a functional component.

### FILED OF THE INVENTION

The present disclosure relates to a method of depositing a fibre onto a freestanding object using a fibre deposition apparatus, and a fibre deposition apparatus, for example an in situ spinning apparatus. The present disclosure also relates to a liquid composition for forming a drawn fibre.

### BACKGROUND

The project leading to this application has received funding from the European Research Council (ERC) under the European Union's Horizon 2020 research and innovation programme (grant agreement No 758865). Sensing elements, for example those used in wearable technology or smart textiles, provide an interface between target objects and the instrumentation used to sense particular aspects of the target object and/or the environment.

Existing fabrication techniques have a number of drawbacks. One drawback of existing sensing elements is the lack of conformability and imperceptibility which results in the sensor itself mismatching or interfering with the target objects. This influences the environment or target object which makes existing sensing elements unfavoured especially for delicate target objects. Furthermore, existing sensing elements are usually produced in a centralised and resource intensive process, which is not environmental-friendly. As an alternative to centralised fabrication, 3D printing, in particular via direct-ink deposition, offers a route for customisable fabrication. However, the sensor interface performance is limited by the thickness of the device needed to fabricate the sensor. The sensor interface performance may also be limited by the network openness of the deposited ink.

The present invention seeks to address at least some of these issues.

### SUMMARY OF THE INVENTION

According to the present disclosure there is provided a method of depositing a fibre onto a freestanding object using a fibre deposition apparatus. The fibre deposition apparatus comprises a dispenser and a moveable arm defining a deposition zone. The method comprises:
positioning the fibre deposition apparatus such that the freestanding object is located within the deposition zone,
forming a droplet of a fibre-forming liquid composition at an outlet of the dispenser,
moving the arm from a first position in contact with the droplet to a second position away from the first position so as to draw the droplet into a drawn fibre, and
depositing the drawn fibre onto the freestanding object as the arm moves from the first position to the second position.

Advantageously, the method provides for imperceptible deposition of the fibres onto the freestanding object because during fibre deposition and does not need to be clamped, constrained or rotated. The freestanding object therefore may not be contacted by the fibre deposition apparatus at all, and only the drawn fibres are in contact with the object. The drawn fibres impart minimal force on the freestanding object, so the deposition is imperceptible and does not disturb, deform or damage the object. Additionally, the object may move naturally during fibre deposition, for example due to wind, vibrations, or natural movement of the object, and the fibres can still be deposited as the arm moves relative to the object. This is particularly advantageous when depositing fibres onto plants or body parts of people because some movement will be inevitable.

Advantageously, controlling movement of the arm relative to the freestanding object allows fibres to be individually deposited onto the freestanding object in a controlled, programmable manner. The fibres may therefore be deposited in a controllable, programmable pattern. For example, the method may provide for depositing fibres in an open array to form a network of fibres on the freestanding object. The open array may be a mesoscopic open array of fibres. Additionally, this is achieved without contacting or clamping the freestanding object, meaning that the fibres can be deposited in an imperceptible manner, allowing the fibre network to be applied to a wide variety of objects without interfering with the objects themselves.

The term “freestanding object” means an object that appears in its original and natural form and is not attached to, or supported by, the fibre deposition apparatus. The freestanding object may be standing by itself, for example a plant or body part. In other examples, the freestanding object may be supported by a different assembly, for example an object held by a person's hand. In examples, a part of the freestanding object may be attached to or supported by the fibre deposition apparatus at a location spaced from the position where fibres are to be deposited. The method may include depositing the fibre directly onto a person's skin.

In examples, the fibre deposition apparatus is an in situ spinning apparatus.

In examples, the fibre deposition apparatus is fibre laying apparatus.

In examples, the fibre is drawn in a wet state, for example a wet sol-gel state. In examples, the drawn fibre is deposited onto the freestanding object in a wet state, for example a wet sol-gel state. Accordingly, the drawn fibre will impart minimal force on the freestanding object and can adhere onto the object due to the wetting of the fibre. This may remove the need for any further processing, such as pressing or gluing, to secure the fibre to the object.

The term “wet sol-gel state” means that the fibre-forming liquid composition remains in a solvent-rich state, where fibre-forming polymers form an inter-connected molecular network through the solvent present in the fibre-forming liquid composition. In some examples, where the fibre-forming liquid composition comprises water as a solvent, the wet sol-gel fibre may have significant wetting, allowing the drawn fibre to adhere onto both hydrophilic and hydrophobic surfaces.

In examples, where the fibre-forming liquid composition comprises polyethylene oxide and water, the wet sol-gel fibre may have significant wetting, allowing the drawn fibre to adhere onto both hydrophilic and hydrophobic surfaces.

In examples, the drawn fibre is deposited onto the freestanding object in a Cassie-like contact state or in a Wenzel-like contact state. Increased fibre “wetness” leads to more contributions from surface tension to form conformal contact (upon fibre drawing), typically leading to Wenzel-like contact states. Generally, in Wenzel-like contact states, the drawn fibres spread out to conform on a rough or textured surface, with increased area of contact, low contact angle and high adhesion. In contrast, non-conformal contract typically leads to Cassie-like contact states. Generally, in Cassie-like contact status, the drawn fibres sit on top of a rough or textured surface with large suspension, reduced area of contact, high contact angle, and low adhesion.

In examples, the arm may comprise a tip that contacts the droplet. The fibre is thereby drawn at the tip. In examples, the arm is sized such that the tip is spaced from the freestanding object during movement of the arm relative to the freestanding object. In examples, the arm, including the tip, does not contact the freestanding object. In examples, no part of the fibre deposition apparatus contacts the freestanding object, only the drawn fibre(s). In examples, the arm is sized such that the tip is spaced from the freestanding object by less than about 5 centimetres, for example less than about 4 centimetres, for example less than about 3 centimetres, for example by between about 1 centimetre and about 3 centimetres. It will be appreciated that the space between tip and the object may change as the arm moves (e.g., if the object has an irregular shape or if the object has some natural movement). The spacing between the tip and the object may be a maximum spacing. A length of the arm may be adjustable for different size objects.

In examples, the arm may be rotated from the first position to the second position. In examples, the arm may be rotated to orbit the freestanding object. In examples, the arm may be rotated at a rate of less than about 100 revolutions per minute, RPM, for example less than about 90 RPM, for example less than about 80 RPM, for example less than about 70 RPM, for example between about 30 RPM and about 65 RPM, for example between about 45 RPM and about 65 RPM, for example about 60 RPM.

In examples, the arm may be rotated about a first axis to orbit the freestanding object. In such examples, the method may further comprise rotating the arm about a second axis different to the first axis. Accordingly, fibres can be deposited with different orientations for forming a network of fibres, or the arm may be selectively rotated about the first or second axis depending on a position and orientation of the freestanding object relative to the fibre deposition apparatus. The first axis may be perpendicular to the second axis.

In examples, the method may further comprise translating the fibre deposition apparatus to generate relative movement between the arm and the freestanding object. In examples, the method may comprise translating the fibre deposition apparatus during movement of the arm between the first position and the second position.

In some examples, the object may be translated relative to the arm, rather than the arm being translated relative to the object. In some examples, both the arm and the object may be translated.

In some examples, the fibre deposition apparatus may comprise a multi-axis robotic arm such as a 3-axis robotic arm, a 4-axis robotic arm, a 5-axis robotic arm, a 6-axis robotic arm or a 7-axis robotic arm. The method may include translating a tip of the robotic arm to deposit the fibre onto the object.

In examples, during depositing of the drawn fibre the freestanding object is stationary. Advantageously, the object does not need to be rotated because the movement of the arm generates relative movement between the arm and the object. This allows the fibres to be deposited on a freestanding object.

In examples, the method may comprise drawing a plurality of fibres and depositing the plurality of fibres onto the freestanding object. The plurality of fibres may be deposited in a network, for example an open array.

In examples, a fibre density of the plurality of fibres deposited onto the freestanding object may be between about 50 fibres per millimetre to about 5 fibres per centimetre. Higher fibre densities can be achieved by adjusting the speed of translation, or by repeating the translation (in the same direction or in reverse) so as to deposit the plurality fibres over the same area of the object more than one time.

In examples, the method may comprise depositing the drawn fibre on a conductive component attached to the freestanding object. The conductive component may be a conductive tape, for example a copper tape. The conductive component and the fibre may together form a functional assembly, for example an electrode or a sensor.

In examples, the method may comprise depositing the drawn fibre to bind a secondary object onto the freestanding object. In examples, the secondary object may be an electronics component. For example, the electronics component may comprise an indicator such as an LED. In this way, the fibre may be used to bind other components to the object and may together form a functional assembly, such as a sensor or electrode. For example, the functional assembly may be an interconnected electronic system.

In examples, the method may comprise depositing one or more fibres onto an object, for example a textile object such as an item of clothing such as a glove. The deposited fibre(s) may alter a surface property of the object, for example to provide electrical conductivity with low skin contact impedance.

In examples, the method may include the step of removing one or more sections of the fibre(s). For example, one or more sections of the fibre(s) may be selectively erased to create openings for electrical connection. In other examples, one or more sections of the fibre(s) may be selectively erased to create openings for circuitry designs and patterning. In some examples, an applicator may be used to physically remove the one or more sections of the fibre(s).

In some examples, the method may include exposing at least part of the fibre(s) to a solvent such as water. At least part of the fibre(s) may be exposed to the solvent prior to or during the step of removing one or more sections of the fibre(s). For example, a controlled humidity chamber may be used to vary a humidity of the environment of the fibre(s). In some examples, solvent may be applied directly to the one or more section of the fibre(s) to be removed. For example, an applicator wetted with solvent may be used to physically remove the one or more sections of the fibre(s). Advantageously, the solvent may reduce the tensile strength of the fibre(s) reducing distortion of the remaining fibre(s) when the one or more sections of the fibre(s) are removed. In some examples, the method may include depositing one or more additional layers onto the fibre(s). The or each additional layer may provide functional integration and/or encapsulate the fibre(s). In some examples, the one or more additional layers may comprise a protective layer.

Beneficially, the protective layer may protect the fibre(s) from abrasion and/or environmental humidity. In some examples, the protective layer may comprise cellulose. For example, the protective layer may comprise cellulose-based fibres and/or a cellulose-based film.

In some examples, the fibre is a first fibre.

The method may include depositing a second fibre onto the first fibre.

The second fibre may be deposited in a similar manner to the that which the first fibre is deposited. The second fibre may have a different composition to the first fibre. In some examples, the second fibre may have the same composition as the first fibre. The second fibre may be deposited in a different pattern to the first fibre. For example, the second fibre may be deposited at an angle with respect to the first fibre.

Depositing the protective layer onto the first fibre may comprise depositing a second fibre onto the first fibre using the fibre deposition apparatus.

The second fibre may comprise cellulose. The first fibre may not comprise cellulose. The second fibre may be water-resistant. Beneficially, cellulose-based fibres may enhance the environmental stability of the sensor. The protective layer may comprise or consist of the second fibre(s).

In examples, the fibre-forming liquid composition comprises nanoparticles, such as silver nanoparticles (AgNP). The method may include sintering the deposited fibre(s). For example, the deposited fibre(s) may be sintered at 100° C. for 15 minutes.

According to a further aspect of the present disclosure there is provided one or more fibres deposited according to the method of any preceding statement. The one or more fibres may form an open array.

According to a further aspect of the present disclosure there is provided a yarn of one or more fibres, formed by the method of any preceding statement, wherein the method involves depositing the one or more fibres onto a support and removing the one or more fibres from the support to form the yarn.

According to a further aspect of the present disclosure there is provided a fibre deposition apparatus for depositing fibres onto a freestanding object. The fibre deposition apparatus comprises:
a dispenser having an outlet for forming a droplet of a fibre-forming liquid composition; and
an arm arranged to move relative to the dispenser from a first position in which the arm contacts the droplet of fibre-forming liquid composition to a second position away from the first position so as to draw the droplet into a drawn fibre,
wherein the arm is arranged to deposit the drawn fibre onto the freestanding object located within a deposition zone defined by the movement of the arm.

Advantageously, the fibre deposition apparatus can be positioned relative to the freestanding object such that the freestanding object is within the deposition zone, without having to clamp, constrain or rotate the object for fibre deposition. This may allow for imperceptible deposition of the fibres onto the freestanding object and can allow for some movement of the object during fibre deposition, as described above.

Advantageously, the arm may be controllable to move relative to the freestanding object to allow fibres to be individually deposited onto the freestanding object in a controlled, programmable manner. The fibres may therefore be deposited in a controllable, programmable pattern. For example, the apparatus may be used for depositing fibres in an open array to form a network of fibres on the freestanding object. The open array may be a mesoscopic open array of fibres. Additionally, this is achieved without contacting or clamping the freestanding object, meaning that the fibres can be deposited in an imperceptible manner, allowing the fibre network to be applied to a wide variety of objects without interfering with the objects themselves. The fibre deposition apparatus may be suitable for depositing fibres directly onto a person's skin.

In examples, the arm is configured to draw the fibre in a wet state, for example a wet sol-gel state. In examples, the fibre deposition apparatus is configured to deposit the drawn fibre onto the freestanding object in a wet state, for example a wet sol-gel state. Accordingly, the drawn fibre can adhere onto the object due to the wetting of the fibre. This may remove the need for any further processing, such as pressing and gluing, to secure the fibre to the object.

In examples, the arm does not contact the freestanding object. In examples, no part of the fibre deposition apparatus contacts the freestanding object, only the drawn fibre(s). In examples, the arm may comprise a tip that contacts the droplet, and wherein the arm is sized such that the tip is spaced from the freestanding object by less than about 5 centimetres, for example less than about 4 centimetres, for example less than about 3 centimetres, for example by between about 1 centimetre and about 3 centimetres. It will be appreciated that the space between tip and the object may change as the arm moves (e.g., if the object has an irregular shape or has some natural movement), and the spacing between the tip and the object may be a maximum spacing. A length of the arm may be adjustable for different size objects.

In examples, the arm may be rotatable from the first position to the second position. The arm may be rotatable to orbit the object. The fibre deposition apparatus may include an actuator, for example a motor, operable to rotate the arm. In examples, the arm may be rotatable at a rate of less than about 100 revolutions per minute, RPM, for example less than about 90 RPM, for example less than about 80 RPM, for example less than about 70 RPM, for example between about 30 RPM and about 65 RPM, for example between about 45 RPM and about 65 RPM, for example about 60 RPM.

In examples, the arm may be rotatable about a first axis to orbit the object, and wherein the arm may be further rotatable about a second axis different to the first axis. The first axis may be perpendicular to the second axis. Accordingly, fibres can be deposited with different orientations to form a network, for example an open array. Additionally or alternatively, the arm may be selectively rotated about the first or second axis depending on a position and orientation of the freestanding object relative to the fibre deposition apparatus.

In some examples, the fibre deposition apparatus may comprise a multi-axis robotic arm such as a 3-axis robotic arm, a 4-axis robotic arm, a 5-axis robotic arm, a 6-axis robotic arm or a 7-axis robotic arm.

In examples, the fibre deposition apparatus may further comprise an actuator operable to translate the arm to generate relative movement between the arm and the object. The fibre deposition apparatus may include an X-Y table operable to move the arm in two perpendicular directions. In examples, the actuator may be operable to translate the arm during movement of the arm between the first position and the second position. In examples, the actuator may be operable to translate the arm such that a fibre density of the plurality of fibres deposited onto the freestanding object is between about 50 fibres per millimetre to about 5 fibres per centimetre.

In examples, the fibre deposition apparatus may include a second arm and/or a second dispenser. The arm may draw fibres from the first and second dispensers to deposit a plurality of fibres onto the freestanding object. Alternatively, the first arm may draw a fibre from the first dispenser, and a second arm may draw a fibre from the second dispenser, and both fibres may be deposited onto the freestanding object. The fibre deposition apparatus may therefore comprise a plurality of arms and/or a plurality of dispensers for depositing a plurality of fibres. The plurality of arms and/or plurality of dispensers may permit deposition of a plurality of fibres simultaneously, and/or deposition of a plurality of fibres with different compositions.

In examples, the fibre deposition apparatus may further comprise a support frame. The arm may be mounted to the support frame. The fibre deposition apparatus may further comprise a coupling arranged to mount the support frame to a stage. The coupling may be arranged to provide at least one degree of freedom of movement between the support frame and the stage so as to allow a user to selectively position the support frame relative to the stage. In examples, the coupling is lockable. In examples, the at least one degree of freedom comprises any of a rotational degree of freedom or a translational degree of freedom.

Advantageously, the stage can be positioned relative to the freestanding object, preferably such that the fibre deposition apparatus is not in contact with the freestanding object, and the position of the support frame can be adjusted and set to position the arm relative to the object.

According to a further aspect of the present disclosure there is provided a liquid composition for forming a drawn fibre. The liquid composition comprises:
a fibre-forming polymer;
a solvent; and
a functional component.

In examples, the liquid composition comprises a long-chain, weakly percolated polymer network.

In examples, the liquid composition (in its non-evaporated state) has a time-dependent shear compliance of between around 10⁻³ to 10⁰ Pa⁻¹ (preferably between around 10⁻² to 10° Pa⁻¹) at the measurement stress of 1 Pa at 25° C.

The weight ratio of the fibre-forming polymer to the functional component may be about 1:1 to 5, preferably about 1:1 to 3, more preferably about 1:1.

In some examples, the volume ratio of the fibre-forming polymer solution to the functional component may be about 1:0.5 to 5, preferably about 1:1 to 3, more preferably about 1:1.

In examples, the fibre-forming polymer comprises one or more polymers selected from the group consisting of polyethers, polycarbonates, polyamides, polyimides, polyolefins, halogenated polymers, aromatic polymers, ketone polymers, methacrylate polymers, polyesters, cellulose derivatives, polypeptides, polysulfones, polyurethanes, polyureas, glycosaminoglycans, polypeptides, polysaccharides, polyphenols, proteoglycans and polynucleotides. In examples, the fibre-forming polymer comprises a polyether and/or an aromatic polymer (e.g., the fibre-forming polymer may comprise polyethylene oxide (PEO), cellulose or its derivatives and/or polystyrene). Preferably, the fibre-forming polymer comprises, or consists essentially of, PEO. In other examples, the fibre-forming polymer comprises a polyether and/or a glycosaminoglycan (for example, the fibre-forming polymer may comprise PEO and/or hyaluronic acid (HA)). Preferably, the fibre-forming polymer comprises PEO and HA. PEO and HA may have particular utility in applications where fibre(s) are deposited onto skin.

In examples, the solvent comprises water and/or an organic solvent. In examples, the organic solvent comprises dimethylformamide (DMF) and/or acetone.

In examples, the functional component comprises a responsive component. In examples, the functional component comprises an electrically-conductive component and/or a colour-responsive component. In examples, the electrically-conductive component is selected from the group consisting of a conductive polymer, conductive carbon particles (optionally carbon nanotubes), metal nanoparticles (for example, silver nanoparticles), metal microparticles and mixtures of two or more thereof. In examples, the electrically-conductive component may be a semi-conducting polymer.

In examples, the fibre-forming polymer comprises PEO, and the functional component is an electrically-conductive component comprising a conductive polymer.

In some examples, the fibre-forming polymer comprises PEO and HA, and the functional component is an electrically-conductive component comprising a conductive polymer.

In examples, the conductive polymer comprises one or more ionomer(s). In examples, the electrically-conductive polymer is poly(3,4-ethylenedioxythiophene) polystyrene sulfonate (PEDOT:PSS). The weight ratio of PEO to PEDOT:PSS may be about 1:1 to 5, preferably about 1:1 to 3, more preferably about 1:1. In a preferred example, the weight ratio of PEO to PEDOT:PSS is about 1:1 to 3.

In examples, the functional component is an electrically-conductive component comprising nanoparticles, such as silver nanoparticles (AgNP).

In examples, the solvent comprises water. In examples, the fibre-forming polymer comprises polystyrene, and the solvent comprises DMF and/or acetone.

In examples, the fibre-forming polymer comprises PEO, and the functional component is a colour-responsive component.

In examples, the colour-responsive component comprises a pH indicator.

In examples, the liquid composition has a total solid mass of less than 10% (w/v), preferably less than 5% (w/v), more preferably less than 3% (w/v).

In examples, the liquid composition is characterised by a ratio of the elastic storage modulus (G′) to the surface tension (γ). The ratio may be between: 0.5 and 3.5; or 1 and 3. Preferably the ratio is between 1.5 and 2.5. The ratio may be calculated using the elastic storage modulus (G′) measured at 2 Hz.

The elastic storage modulus (G′) may be measured by parallel plate shear creep at 25° C. with a 1 Pa stress input.

According to a further aspect of the present disclosure there is also provided a method, as described above, wherein the fibre-forming liquid composition is a liquid composition as described above.

According to a further aspect of the present disclosure there is also provided a fibre deposition apparatus, wherein the fibre-forming liquid composition is a liquid composition as described above.

According to a further aspect of the present disclosure, there is also provided a sensor comprising a plurality of fibres drawn from a liquid composition and deposited onto a freestanding object.

In examples, the plurality of fibres circumscribe the freestanding object.

The fibre-forming liquid composition may be the liquid composition according to any preceding statement.

In examples, the sensor further comprises a conductive element and/or an electronics component located between the freestanding object and the plurality of fibres. For example, the electronics component may comprise an indicator such as an LED. Such an indicator may be used in crop management systems.

In examples, the sensor further comprises a connector for connection to a sensor unit.

In examples, the sensor is operable to detect one or more of:
electrocardiography, ECG, data
electromyography, EMG, data,
surface texture,
surface material type,
a pH of the freestanding object or the environment surrounding the freestanding object,
a concentration or presence of a target species of the freestanding object or the environment surrounding the freestanding object,
movement or transformation of the freestanding object,
temperature of the freestanding object,
pressure applied to the freestanding object, and/or
a strain of the freestanding object.

In examples, the sensor is operable to detect surface textures. For example, the sensor may be operable to detect surface textures by piezoelectrically generated charges.

In examples, the sensor may be operable to detect surface textures by triboelectrically generated charges.

For example, the sensor may be operable to detect a smooth surface and/or surfaces defined by: a wave profile, a square wave profile, and/or a sawtooth profile.

In examples, the sensor may be operable to detect a surface texture of various different materials such as wood and/or plastics such as Polyethylene terephthalate (PET), Polytetrafluoroethylene (PTFE), and/or synthetic rubbers (e.g., Ecoflex).

In examples, the sensor is operable to characterise surface materials. For example, the sensor may be operable to detect surface material type by triboelectrically generated charges.

In examples, the sensor may be operable to detect surface material type by piezoelectrically generated charges.

The sensor may be operable to determine a material composition of a surface. For example, the sensor may be operable to characterise a surface material as wood and/or plastics such as Polyethylene terephthalate (PET), Polytetrafluoroethylene (PTFE), and Ecoflex

According to a further aspect of the present disclosure there is also provided a method of characterising a surface using a sensor such as any sensor described herein. The method may include periodically tapping fibres of the sensor against a surface and recording an output (e.g. an output voltage) indicative of the material type and/or composition. The method may include sliding fibres of the sensor along a surface and recording an output (e.g. an output voltage) indicative of the surface texture.

The sensor may be operable to detect vapour present in the environment surrounding the plurality of fibres. For example, the vapour may be water vapour or ammonia vapor. The sensor may be operable to detect the presence of the vapour and/or the concentration of the vapour and/or the chemical composition of the vapour.

The sensor may be operable to measure fluid, such as water droplets, contacting the plurality of fibres. In examples, the sensor may be operable to detect the presence or absence of water droplets, and/or the number or amount of water droplets, and/or the chemical composition of the fluid.

According to a further aspect of the present disclosure, there is also provided a device for heat generation or refrigeration and/or energy harvesting comprising a plurality of fibres drawn from a liquid composition and deposited onto a freestanding object.

In examples, the plurality of fibres circumscribe the freestanding object.

In examples, the plurality of fibres comprise a thermoelectric element.

In examples, the liquid composition comprises nanoparticles, such as silver nanoparticles (AgNP). In examples, the plurality of fibres comprise nanoparticles, such as silver nanoparticles (AgNP).

An object comprising a plurality of fibres deposited thereon, wherein the plurality of fibres form an open array of fibres.

The plurality of fibres may be configured as a regular array. For example, the regular array may be in the form of evenly spaced parallel lines, a square matrix, a fanning array, or a parallelogram array.

The plurality of fibres may be configured as an irregular array. For example, the irregular array may be in the form of a programmable trace.

The object may comprise a textile and the plurality of fibres may be deposited onto the textile. The textile may be a woven or non-woven collection of natural or synthetic fibres. For example, the textile may be a yarn or natural fibres or human hair.

According to a further aspect of the present disclosure, there is also provided an object comprising a fibre deposited thereon, wherein the fibres are morphed to the surface topography such that the fibre is in contact with the surface topography at over 20%, 30%, 40% or 50% of its length when observed at a microscopic scale.

In some examples, the fibre is deposited onto the object while the fibre is in a wet state.

In some examples, the microscopic scale is a range of 100-1 μm, 50-1 μm, 10-1 μm at 100 μm scale.

In some examples, the fibre does not comprise a volatile solvent.

In some examples, the fibre does not comprise PEO.

In some examples, the liquid-fibre composition from which the fibre was drawn does not comprise PEO. In some examples, the liquid-fibre composition from which the fibre was drawn does not comprise a volatile solvent.

In some examples the fibre is an electrically-conductive fibre. For example, the electrically-conductive fibre may have the composition of any electrically-conductive fibre described herein. For example, the electronically-conductive fibre may have a composition according to any preceding statement.

In some examples, the object comprises organic matter and the fibre is deposited on the organic matter.

In some examples the organic matter is skin or plant matter.

In some examples, the object comprises a plurality of fibres deposited thereon, wherein each of the plurality of fibres are morphed to the surface topography such that each of the plurality of fibres are in contact with the surface topography at over 20%, 30%, 40% or 50% of each of the plurality of fibres length when observed at a microscopic scale.

In some examples each of the plurality of fibres is an electrically-conductive fibre. For example, the electrically-conductive fibres may have the composition of any electrically-conductive fibre described herein. For example, the electronically-conductive fibres may have a composition according to any preceding statement.

According to a further aspect of the present disclosure, there is provided an object comprising one or more fibres deposited thereon. The fibres may be characterised by a maximum peeling force per unit fibre width of over 15 N, when measured according to a 90-degree peeling test.

The fibres may be characterised by a maximum peeling force per unit fibre width of between 5 N and 15 N, between 10 N and 15 N, between 5 N and 17N, between 10 N and 17N (when measured according to a 90-degree peeling test).

The peeling test may be defined by the ASTM D2861 90-degree peeling test.

The peeling test may be carried out on fibres deposited as evenly spaced parallel lines oriented perpendicular to a lifting arm.

In some examples each of the fibres is an electrically-conductive fibre. For example, the electrically-conductive fibres may have the composition of any electrically-conductive fibre described herein. For example, the electronically-conductive fibres may have a composition according to any preceding statement.

### BRIEF DESCRIPTION OF FIGURES

Embodiments of the invention are further described hereinafter with reference to the accompanying drawings, in which:

FIG. 1 is a schematic representation of an exemplary fibre deposition apparatus;

FIG. 2 illustrates a first exemplary application of the apparatus of FIG. 1;

FIG. 3 illustrates data from the application of FIG. 2;

FIG. 4 illustrates a further exemplary application of the apparatus of FIG. 1;

FIG. 5 illustrates the impedance characteristics of a sensing element;

FIG. 6 illustrates ECG data captured using a sensing element compared to gel electrodes;

FIG. 7 illustrates further impedance characteristics of a sensing element;

FIG. 8 illustrates exemplary ECG data captured using a sensing element at different time points and under different moisture conditions;

FIG. 9A to 9B illustrate stretching characteristics of a fibre array;

FIGS. 10A to 10C illustrate shear compliance data of bioelectronic fibre solutions (i.e., electrically-conductive fibres) and pH-responsive fibre solutions;

FIGS. 11A & 11B illustrates exemplary ECG data captured using a sensing element at different dry friction times;

FIG. 12 illustrates a tensile testing apparatus for characterising a fibre array;

FIGS. 13A to 13B illustrate tensile properties of two fibre arrays under varied humidity conditions;

FIGS. 14A to 14B illustrate results of scratching tests for fibre contacts with and without a protective layer;

FIGS. 14C and 14D illustrate results of friction tests for fibre meshes with and without a protective layer;

FIGS. 15 to 16 illustrate further exemplary applications of the apparatus of FIG. 1;

FIG. 17 illustrates exemplary mechanical property data of exemplary fibres made using the apparatus of FIG. 1.

FIG. 18 illustrates a further exemplary application of the apparatus of FIG. 1;

FIGS. 19 to 21 illustrate exemplary EMG data captured using a sensing element under different loading conditions;

FIG. 22A illustrates a further exemplary application of the apparatus of FIG. 1;

FIG. 22B illustrates exemplary data captured from the application of FIG. 22A;

FIG. 23A illustrates a further exemplary application of the apparatus of FIG. 1;

FIG. 23B illustrates exemplary data captured from the application of FIG. 23A;

FIGS. 24A & 24B illustrate further exemplary applications of the apparatus of FIG. 1;

FIG. 25 illustrates exemplary data captured from the application of FIGS. 24A & 24B;

FIG. 26 illustrates a further exemplary application of the apparatus of FIG. 1;

FIG. 27 illustrates exemplary data captured from the application of FIG. 26;

FIGS. 28A to 28B illustrate further exemplary data captured from the application of FIG. 26;

FIGS. 29A & 29B illustrate exemplary data captured from an application of the apparatus of FIG. 1;

FIG. 30A illustrates a further exemplary application of the apparatus of FIG. 1;

FIGS. 30B & 30C illustrate exemplary data captured from the application of FIG. 30A;

FIG. 31 is a further exemplary fibre deposition apparatus;

FIG. 32 is a further exemplary fibre deposition apparatus;

FIGS. 33A & 33B illustrate a further exemplary fibre deposition apparatus; and

FIG. 34A illustrates elastic storage modulus (G′) data for various solutions;

FIG. 34B illustrates surface tension (γ) data for the solutions of FIG. 34A;

FIG. 34C illustrates data representing a ratio of G′ to γ for the solutions of FIG. 34A;

FIG. 35A to 35C illustrate an object with a plurality of electrically-conductive fibres deposited thereon;

FIG. 36 illustrates an object with a plurality of PEO-based fibres deposited thereon;

FIGS. 37A & 37B illustrate results of surface adhesion characterisation of an electrically-conductive fibre array; and

FIG. 38 illustrates results of voltage measurements of an electrically-conductive fibre array taken during surface impact experiments for a variety of different surface materials; and

FIG. 39A illustrates results of surface texture characterisation using an electrically-conductive fibre array; and

FIG. 39B illustrates a variety of different surface textures tested in the surface characterisation of FIG. 39A.

### DETAILED DESCRIPTION

The present apparatus allows for imperceptible deposition of sensing fibre patterns that adaptively morph onto target objects, for example living or soft structures. The sensing fibre patterns can be created in minutes at the point-of-use as explained below. The fibre sensing elements described herein can be inbuilt with bioelectronic or colorimetric readouts, enabling augmented perceptions, dual-model sensing, and wearables-for-plants, without restricting key biological functions of the living structures. The networks formed by the apparatus advantageously are provided using substrate-free fibres which minimises any interference to the host, while supporting integration with microelectronics and e-textiles, enabling on-demand and efficient fibre repairs, upgrades, and recycling when the fibre device needs to be reconfigured.

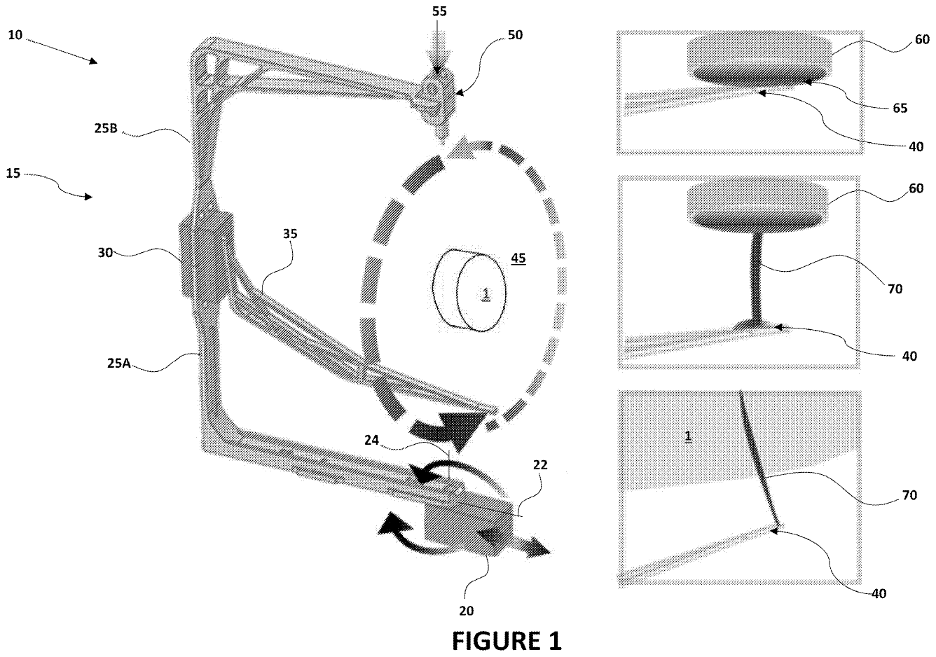

FIG. 1 illustrates an exemplary fibre deposition apparatus 10 using an in situ spinning process. The apparatus 10 includes a support frame 15, a coupling 20 mounted to the support frame 15, a dispenser 50 mounted to the support frame 15 and an arm 35 connected to the support frame 15 via a motor 30 which rotates the arm 35 about an axis of rotation. As the arm 35 rotates, the trajectory of the arm, in particular a tip 40 of the arm 35, circumscribes a deposition zone 45 into which a freestanding object 1 may be placed so that fibres can be deposited onto the surface of the freestanding object 1. The dispenser 50 dispenses the fibre-forming liquid composition 65 in a controlled manner at an outlet port 60. The outlet port 60 is at the bottom of the dispenser 50 and causes the fibre-forming liquid composition 65 to accumulate on the outlet port 60 as shown in the upper right panel of FIG. 1.

As the arm 35 rotates, the tip 40 will contact the fibre-forming liquid composition 65 accumulated on the outlet port 60, and draw the fibre-forming liquid composition 65 into a fibre 70 (middle right panel in FIG. 1). As the tip 40 moves away from the outlet port 60 and around the freestanding object 1 in the deposition zone 45, the fibre 70 will be deposited on the object 1, before returning to the initial position where it contacted the fibre-forming liquid composition 65 on the outlet port 60 to draw a new fibre. This process is repeated until sufficient fibres 70 have been wound around the object 1 to provide a functional sensing element 75. The fibres 70 are drawn in a wet state, in particular in a wet sol-gel state (i.e., a solvent-rich state) around the target. When an object is placed within the deposition zone 45, the contactless deposition of a lightweight, wet fibre 70 induces minimal disturbance on the object 1. This is seemingly imperceptible to the object, which may be a living object such as a plant or person. In some cases, the deposition zone is cylindrical. In some cases, the deposition zone has a depth up to 300 mm (taken in the direction of the rotation axis of the arm 35), which accommodates objects 1 having a size of between less than a millimetre to tens of centimetres.

In the illustrated example, the dispenser receives a fibre-forming liquid composition 65 through an inlet port 55 from an external reservoir of fibre-forming liquid composition 65. However, the inlet port 55 is not essential, as the dispenser may have an internal reservoir of fibre-forming liquid composition 65. While the dispenser 50 is shown having a single outlet port 60, it would be apparent that the dispenser 50 may have multiple outlet ports 60 to produce multiple fibres 70 at the same time, or that multiple dispensers 50 may be provided to dispense a plurality of the same or combinations of different fibres 70 as required.

In examples, the apparatus requires approximately 5 W to run, produces no CO₂ or toxic emissions, and only consumes 60 μL per hour of fibre-forming liquid composition 65 to create sensing elements 75. This is considerably more ecological than existing systems designed for fibre spinning processes. Typical fibre networks created within 2-5 minutes will require a total solution usage of approximately 5 μL and a dry mass input of approximately 0.1-0.3 mg per fibre network.

While the support frame 15 is made up of two frame sections 25A, 25B it would be apparent this was not essential. It is also not essential for the frame sections 25A, 25B to be made up of L-shaped sections. While the arm 35 is arranged to move in a rotary manner, it would also be apparent this was not essential, and in some cases, the fibres may be drawn and wound around the object 1 using a combination of linear motions. While the tip 40 is described as contacting the fibre-forming liquid composition 65 this is also not essential, as other parts of the arm 35 could contact the fibre-forming liquid composition 65 and draw a fibre 70. In one example, the arm 35 may move additionally or alternatively in a linear manner to deposit fibres in a pre-determined pattern on the target 3. For example, a linear actuator may translate the arm 35 at 50 μm/s.

The present apparatus 10 allows for fibres 70 to be deposited directly onto a target freestanding object 1 to provide a biointerface sensing elements. The apparatus 10 deposits fibres 70 adaptively, negligibly impacting its environments, and the fibres 70 can be maintained and adjusted as required, or disposed at the end of use due. The present apparatus 10 requires considerably less starting material and the sensing element 75 can degrade naturally, creating eco-adaptive sensing elements with ultra-low embodied emissions, materials. The orbital solution spinning approach of the present apparatus 10 provides microfibres, which adaptively ‘morph’ around the periphery of a target freestanding object, thus eliminating the need to provide a digital map of the target object 1. As the fibres 70 morph according to the surface features of the object 1, this makes the present apparatus tolerant of slight movement of the object 1 within the deposition zone 45, as may often be present in real world applications. When a stationary object 1 is placed within the deposition zone 45, stepped movements of the arm 35 produce designable fibre patterns with a spacing resolution down to for example around 50 μm and an inclination angle of up to around 50 degrees relative to the axis of rotation. The stresses imposed by the fibres 70 is in the order of tens of Pascals. Such low stresses are typically imperceptible to the object 1 and thus do not interfere with any biological processes the object (e.g., a plant or animal) may be undergoing.

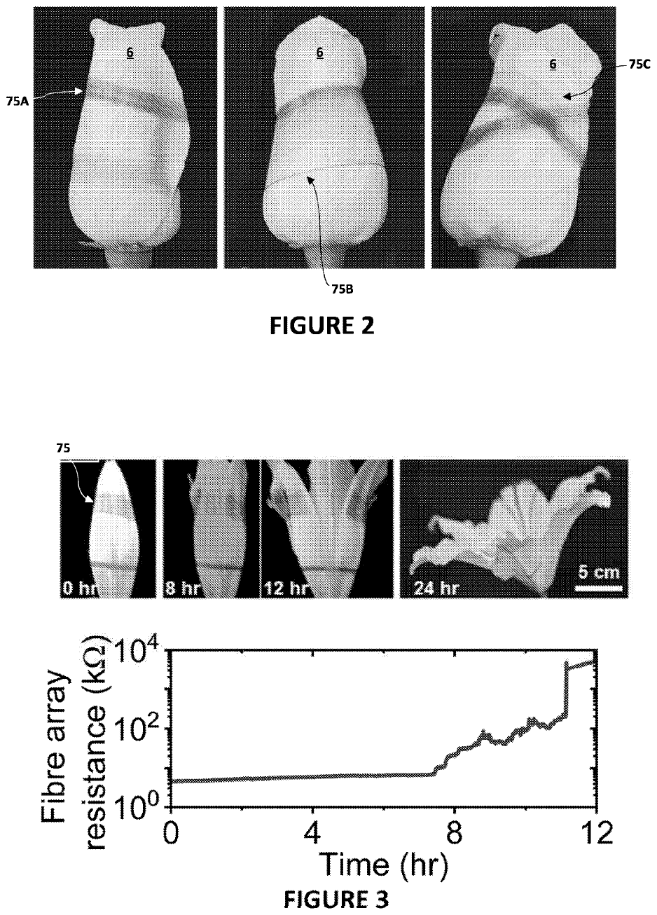

The coupling 20 is not essential to the apparatus. When present, the coupling 20 provides additional degrees of freedom between the support frame 15 and a stage (not shown). The coupling 20 allows the support frame to rotate and/or translate about axes 22 and 24 shown in FIG. 1. In one example, the support frame 15 can rotate about axis 24 (a substantially vertical axis) and translate along axis 22 (a substantially horizontal axis). Axes 22 and 24 are substantially orthogonal to each other. The dispenser 50 is shown directly above the coupling 20, but this would not be essential. In some cases, the axis of rotation is parallel to axis 22. By varying the position of the support frame 15 about axes 22 and 24, it is possible to provide sensing elements 75A, 75B, 75C shown in FIG. 2. By setting the arm 35 movements accordingly, it is possible to produce evenly distributed, on-off, or graded fibre arrays as shown in FIG. 2. The coupling 20 can be manually adjusted to a user-defined position or orientation relative to any of axes 22 and 24. In some cases, the support frame 15 may be actuated by an actuator (not shown) along or about any of axes 22 or 24.

In an example use, the fibres are deposited on plants to measure the development of the plants without interfering with the plants or their development. As plants can undergo rapid transforms in shapes and forms, any sensing elements 75 directly interface a plant's surface should be designed to minimally interfere with the plant's intrinsic biological processes and functions. FIGS. 2 and 3 illustrate a lily with a sensing element 75 designed to measure when the lily flower 6 opens. By winding a sensing element 75 around the base of the lily flower 6, and as the lily flower 6 opens this will gradually break the fibres 70 of the sensing element 75, which can be measured using an external circuit as shown in FIG. 3. In an example, the force required to break each fibre 70 made of PEDOT:PSS is typically between 50 to 200 μN. Based on the force consideration for fibre breakage, the fibre sensing element 75 can be designed as shown in FIGS. 2 and 3 to accommodate the opening process of lily flowers. While a lily is shown as an exemplary freestanding object, the present fibres sensing elements 75 are applicable to a wide range of applications, such as in-situ crop monitoring, pollination and selective insect control (e.g., by altering recipes and adding specific chemicals).

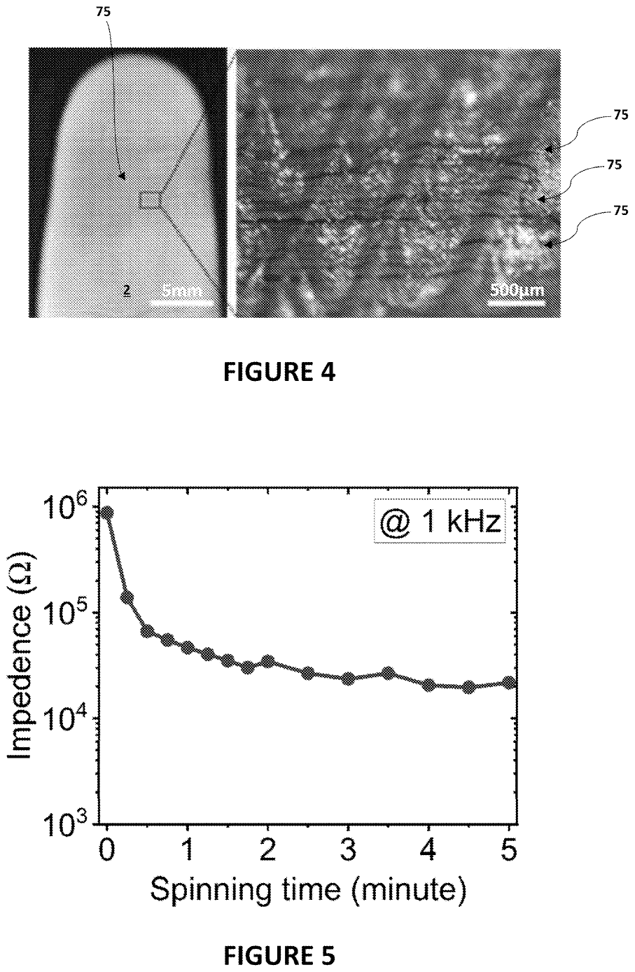

FIG. 4 illustrates fibres 70 being wound around a finger 2 to provide a fibre array 75 for sensing of biophysiological signals, (e.g., electrocardiogram (ECG) and/or electromyogram (EMG) signals). A second finger on the other hand (not shown) was also instrumented using the same technique to provide a reference electrode. In both cases, an array of fibres were wound around the fingertip. A strip of copper tape is placed onto the nail to serve as connection before thread printing. FIG. 5 shows the impedance of the sensing element of FIG. 4 as the fibres 70 are wound around the finger 2 at a rate of approximately one revolution per second. As shown in FIG. 5, after 2 min of deposition at the rate of one fibre loop per second, the contact impedances of the fibre array on the skin of an index finger decreased to 34 kΩ at 1 KHz, a value comparable to reported microfabricated gold nanomesh. In some cases, the impedance is approximately 50 kΩ at 1 KHz Thus the impedance of the in-situ sensing element 75 can use be used to collect data in a range of applications after a few minutes of deposition. Consistent impedance was measured across different fibre sensing element 75 experiments and ambient deposition environments as explained below. It would be apparent that the amount of material deposited onto an object will vary depending on the application, but fibre sensing elements 75 weighing 10 μg have been found to be effective.

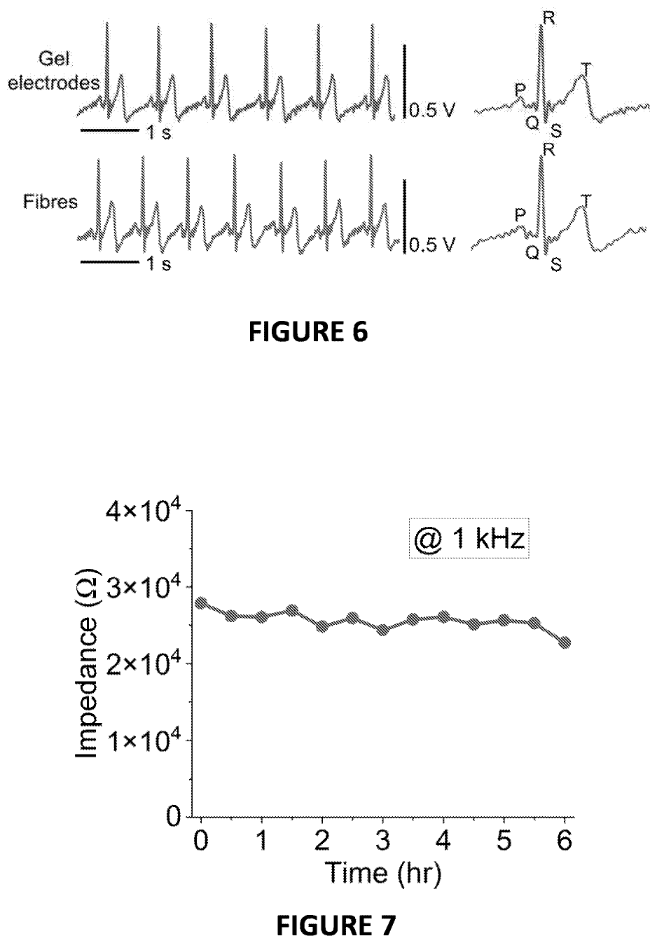

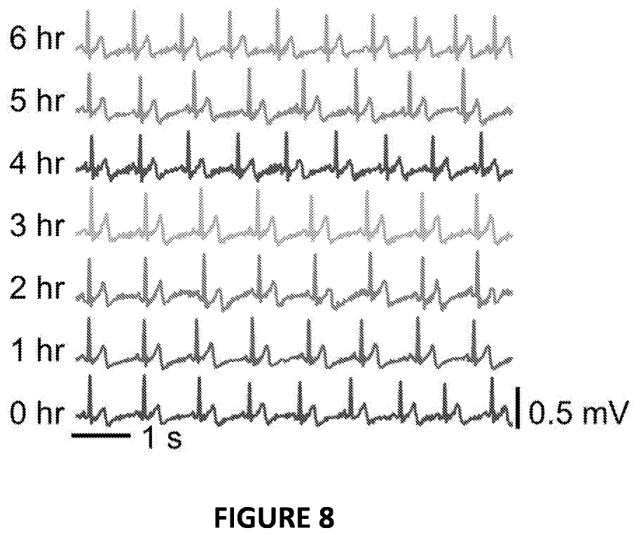

As shown in FIG. 6, the sensing element 75 of FIG. 4 provides comparable signals to gel electrodes which are typically used in ECG data collection. As shown on the right hand side of FIG. 6, the QRS complex from the fibre-based ECG data are highly distinct. The present fibres 70 are also highly robust to environmental and mechanical wear. As shown in FIGS. 7 and 8, even with prolonged wear during normal activities of daily living and after repairing the electrode following deposition of further fibres, the impedance of the sensing element 75 remained relatively consistent, and it as possible to capture longer term ECG data over a range of points during a continuous 6 hour period.

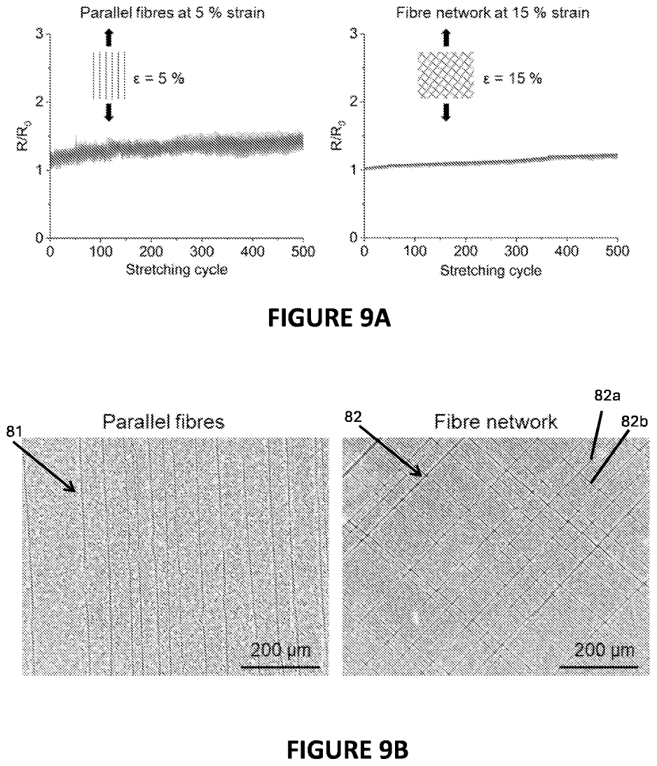

FIG. 9A illustrates stretchability characterisation of a parallel fibre array 81 and an orthogonal fibre array 82. Images showing the two fibre arrays 81, 82 tested are shown in FIG. 9B. As shown, the parallel fibre array 81 comprises a single set of parallel fibres while the orthogonal fibre array 82 comprises a first set of parallel fibres 82a and a second set of parallel fibres 82b. The first set of parallel fibres 82a are orthogonal to the second set of parallel fibres 82b.

The fibre arrays 81, 82, were deposited onto an elastomer film (~1 mm thickness, Ecoflex 00-30). The samples were clamped and cyclically stretched using a motorised stage (Thorlabs MTS50-Z8). As shown in FIG. 9A, the parallel fibre array 81 was stretched parallel to the fibres (ε=0 to 5%) while the orthogonal fibre array 82 was stretched at 45° to the first set of parallel fibres 82a (i.e., −45° with respect to the second set of parallel fibres 82b) (ε=0 to 15%).

The resistance of the fibre arrays 81, 82 was recorded using a multimeter (Keysight 34465A). As shown in FIG. 9A, the normalised resistance of the parallel fibre array 81 remained relatively constant over hundreds of stretching cycles for strains of 5%. The orthogonal fibre array 82 showed even lower resistance variation even at a higher strain of 15%. The ability of the fibres to withstand repeated stretching is beneficial in many applications. For example, stretchability may allow the fibres to be imperceptible when deposited onto a flexible surface such as skin which is subject to repeated stretching.

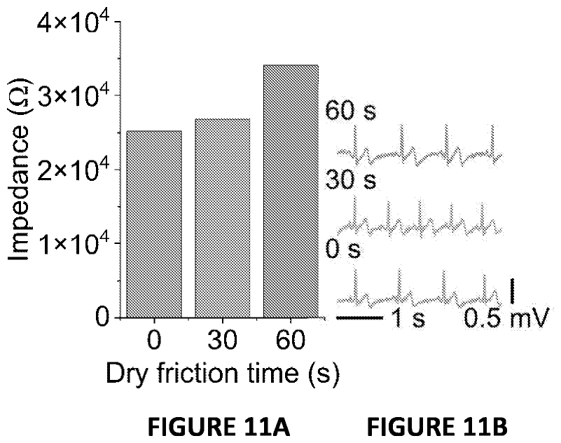

Repairability is a further advantage of tethering individual fibre building blocks, as compared to existing microfabricated interface with a monolithic or fully-integrated architecture. For example, when a tethered fibre array is deliberately damaged by abrasion, new fibres could be deposited on demand without affecting existing interconnections. The biopotential acquisition interface can be fully renewed with considerably less starting material compared to what was needed for the initial deposition. FIGS. 11A to 11B further illustrate the utility of the sensing element 75 after mechanical rubbing for different durations of time, the contact impedances and ECG signals measured on an index finger via bioelectronic fibres, subjected to dry rubbing for different durations. An index finger with fibres deposited was put to rub on an A4 paper, with normal force of around 2 N at a frequency of around 1 Hz for 30 seconds and 60 seconds. As shown in FIGS. 11A and 11B, the attached bioelectronic fibres remain fully functional following typical daily movements and activities.

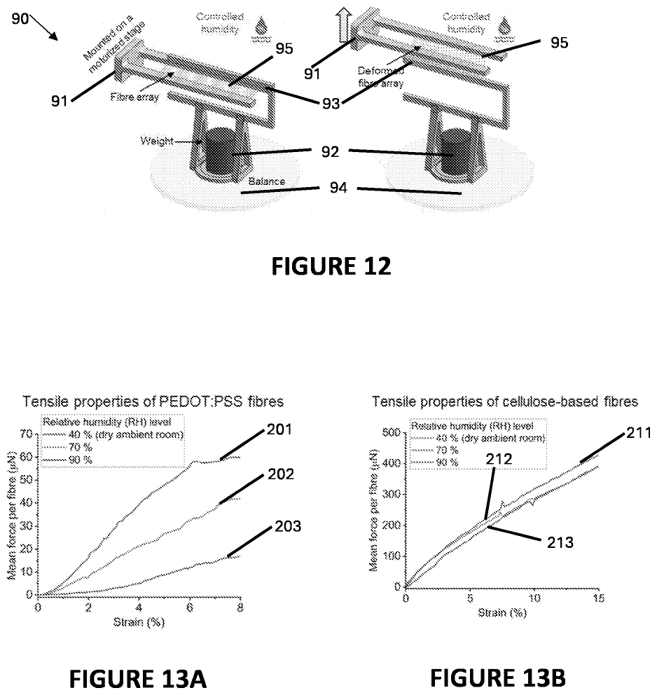

The tensile properties of the fibres may be affected by environmental factors such as humidity. This effect is illustrated by FIGS. 12 to 13B which show how the tensile properties of two different fibre compositions vary with humidity. FIG. 12 shows a method 90 for testing the tensile properties of the fibres. As shown, the fibres are looped around two rigid tines of a motorised stage 91 (vertical translational stage: Thorlabs MTS50-Z8) to form an array 95 of parallel fibres. In this example, the tines are spaced apart by 20 mm. A weight 92 is seated on a balance 94 (Ohaus Scout Portable Balance, 120 g Capacity, 0.001 g Readability) such that a hook 93 of the weight 92 is centred above the two tines. As the motorised stage 91 is raised, the hook 93 contacts the array 95 which deforms to accommodate the hook 93. The reading on the balance 94 reflects the force induced by the deformed fibre array 95. The strain of the fibre array 95 was calculated using equation 1 below where D is the displacement of the motorised stage 91 and L is the original length of the fibre array 95.

$$Strain = \frac{2 \sqrt{{D}^{2} + {( \frac{L}{2} )}^{2}} - L}{L} \qquad Equation 1$$

A controlled humidity chamber (not shown) was used to control the humidity of the environment surrounding the array of fibres 95.

FIG. 13A shows a plot of the mean force per fibre against strain for PEDOT:PSS fibres. Results are shown for 40% relative humidity 201, 70% relative humidity 202, and 90% relative humidity 203. As shown, the tensile strength of the PEDOTT:PSS fibres diminishes significantly with increasing environmental humidity.

FIG. 13B shows a plot of the mean force per fibre against strain for cellulose fibres. Results are shown for 40% relative humidity 211, 70% relative humidity 212, and 90% relative humidity 213. As shown, the cellulose-based fibres possess almost 10 times higher tensile strength than the PEDOT:PSS fibres. Additionally, the tensile strength of the cellulose-based fibres was found to be largely independent of the environmental humidity. The applicant has appreciated that a cellulose-based protective layer may be used to protect the bioelectronic fibres (e.g., PEDOT:PSS based fibres). For example, the protective layer may comprise cellulose-based fibres which encapsulate the bioelectronic fibres. The cellulose-based fibres may be deposited onto an exterior surface of the bioelectronic fibres using the apparatus and method described herein. Beneficially, the cellulose-based fibres may enhance the environmental stability of the fibres. In some examples, the protective layer may comprise both cellulose-based fibres and a cellulose film which overlies the cellulose-based fibres.

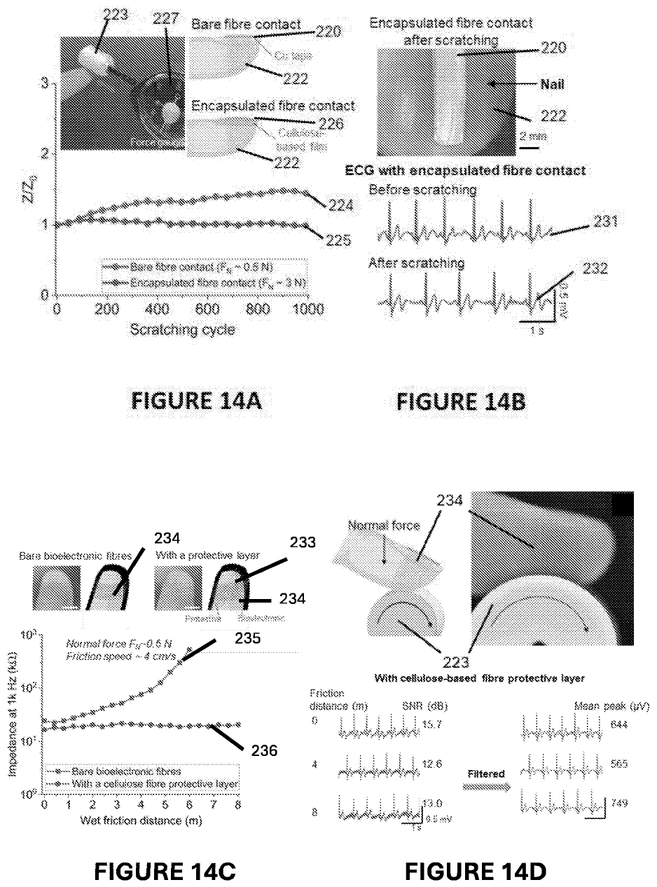

The utility of the protective layer is highlighted by FIG. 14A which shows results of scratching tests for fibre contacts with and without a protective layer. In this example, the protective layer is a cellulose film which overlies the fibre contact. The experimental setup is shown at the top of FIG. 14A. As shown, a Cu electrode 220 is attached to a fingernail 221 of a participant. Bio-electric fibres 222 were deposited around the fingertip, overlying the Cu electrode 220. For the encapsulated fibre test, a cellulose film 226 was deposited on top of the bio-electric fibres 222 to cover the Cu electrode 220. A control was also performed in which the bio-electric fibres 222 were left exposed.

During scratch testing, an abrasive fabric arm 223 was oscillated substantially parallel to the fibres 222 while the impedance of the electrode was measured. The abrasive fabric arm 223 was fixedly connected to a force gauge 227 to maintain a fixed abrasive force during the scratch test. The normalised impedance results for the control 224 and test 225 conditions are shown at the bottom of FIG. 14A. As shown, the impedance of the control 224 increased with the number of scratch cycles even at a low abrasive force of 0.5 N. In contrast, the impedance of the test condition 225 (where the protective cellulose layer was provided) did not increase with the number of scratch cycles even at a moderate abrasive force of 3 N.

FIG. 14B shows the encapsulated fibre contact following 1000 scratching cycles at 3 N. As shown, the fibres 222 in the encapsulated fibre test were still present and effective after 1000 scratching cycles at 3 N. As previously described in relation to FIGS. 4 to 6, an array of fibres deposited onto a finger may be used as a sensing element to detect ECG signals. A plot of measured potential against time for the encapsulated fibre contact (sensing element) is provided at the bottom of FIG. 14B. ECG results recorded prior to the scratching test are illustrated by line 231 while ECG results recorded following 1000 scratching cycles at 3N are illustrated by line 232. As shown, even after significant abrasion, an ECG signal can still be clearly detected by the encapsulated fibre contact. Beneficially the protective layer may prolong the lifetime of the fibres and minimise their susceptibility to damage caused by abrasion, especially in humid conditions. FIGS. 14C and 14D show results of friction tests for fibre meshes with and without a protective layer. In this example, the protective layer comprises cellulose-based fibres 233. As shown in the top of FIG. 14C, the cellulose-based fibres 233 have deposited onto an exterior surface of the bioelectronic fibres 234 (using the apparatus and method described herein) and encapsulate the bioelectronic fibres 234. In this example, the cellulose-based fibres 233 were deposited in a 9 mm wide array comprising ~90 fibres. The cellulose-based fibres 233 were oriented at 30° relative to the bioelectronic fibres 234. Angling the cellulose-based fibres 233 relative to the bioelectronic fibres 234 may further enhance the protective capabilities of the protective layer.

The experimental setup shown at the top of FIG. 14D was used to apply friction to bioelectronic fibre arrays with and without the encapsulating cellulose-based fibres 233. During friction testing, the abrasive fabric arm 223 was rotated at a speed of 4 cm/s substantially perpendicular to the bioelectronic fibres 234 while the impedance was measured. The abrasive fabric arm 223 was connected to a force gauge 227 to maintain a fixed abrasive force of 0.5N during the friction test. The normalised impedance results for the control 235, having no protective layer, and the test 236 having the cellulose-based fibres protective layer, are shown at the bottom of FIG. 14C. Both the control 235 and the test 236 were wetted with water prior to testing to record friction properties under wet conditions. As shown, the impedance of the control 235 increased exponentially with the wet friction distance. In contrast, the impedance of the test 236, where the protective cellulose-based fibres 233 were provided, did not increase significantly with the wet friction distance.

FIG. 14D shows ECG signals detected using a fibre array with the encapsulating cellulose-based fibres 233. Results correspond to measurements taken prior to the wet friction test (Om friction distance) as well as after 4 m and 8 m friction distance. As shown, the fibre array with the encapsulating cellulose-based fibres 233 were still present and effective after 8 m friction distance. As shown, even after significant abrasion, an ECG signal can still be clearly detected by the encapsulated fibre array. Beneficially the protective layer may prolong the lifetime of the fibres and minimise their susceptibility to damage caused by abrasion, especially in humid or wet conditions.

By tuning the chemical composition of the fibre-forming liquid composition 65, it is possible to provide fibres with certain mechanical properties. For example, fibres 70 can be formed which are entirely biocompatible or biodegradable organic-based compositions which are further tailored to the types of living structures to be interfaced. The fibre-forming liquid composition comprises a fibre-forming polymer; a solvent; and a functional component. Any suitable fibre-forming polymer can be used in the fibre-forming composition. In some cases, the fibre-forming polymer is polyethylene oxide (PEO) which acts as a base to facilitate fibre drawing. PEO is preferable, as it is widely present as a food and drug additive.

For skin-interfaced bioelectronic sensing, electrically-conductive fibres would be suitable. For example, the functional component can be poly(3,4-ethylenedioxythiophene) polystyrene sulfonate (PEDOT:PSS), which is a biocompatible polymer with mixed conductivity. The fibre-forming liquid composition may further comprise hyaluronic acid (a major extracellular matrix component of skin).

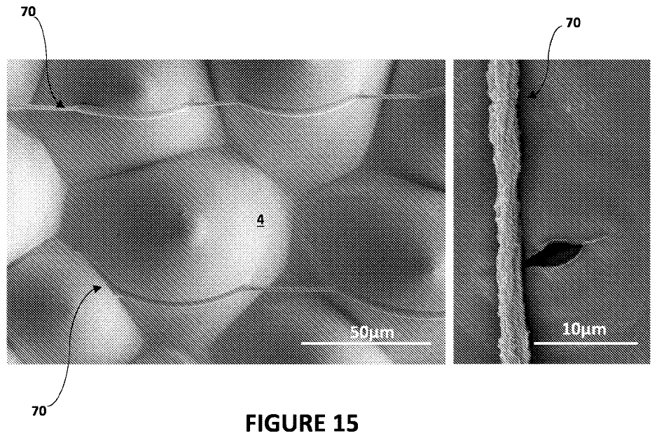

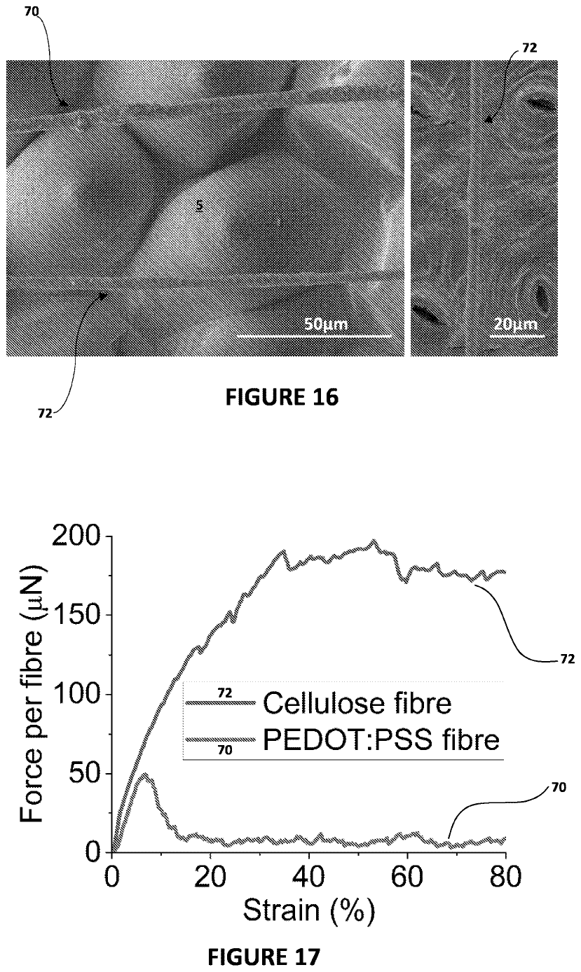

For environmental sensing, colour-responsive fibres (e.g., pH responsive fibres) would be suitable. For example, the functional component may be a responsive component and may comprise a pH indicator. The fibre-forming composition may further comprise cellulose nanofibers and pH responsive dyes for pH sensing applications. By way of example, the colour-responsive fibres (e.g., pH-responsive fibres 72) comprising cellulose nanofibers and pH responsive dyes can be made stiffer than the electrically-conductive fibres 70 comprising PEDOT:PSS, as shown in FIG. 17. FIG. 15 shows the electrically-conductive fibres 70 comprising PEDOT:PSS conforming to the cells of an orchid. In contrast, FIG. 16 shows the colour-responsive fibres 72 suspended above the cells of a leaf around a stomata of the leaf due to the non-conforming nature of the stiffer colour-responsive fibres 72. In comparison to painting or spraying of films and fibres, the present fibres 70, 72 produce small and designable aerial mass density of the fibre array 75. Such open fibre frameworks minimally conceal any surface receptors and pores exposed on the living structures.

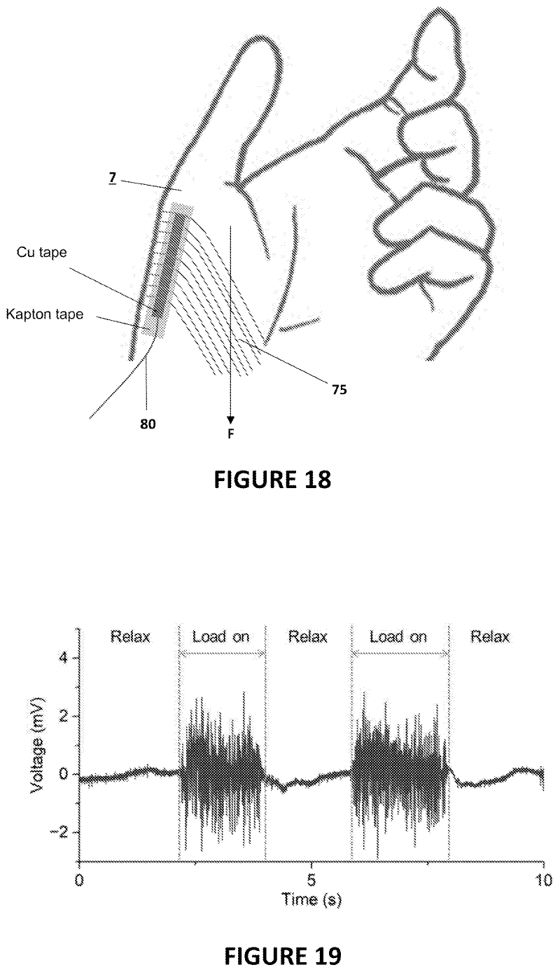

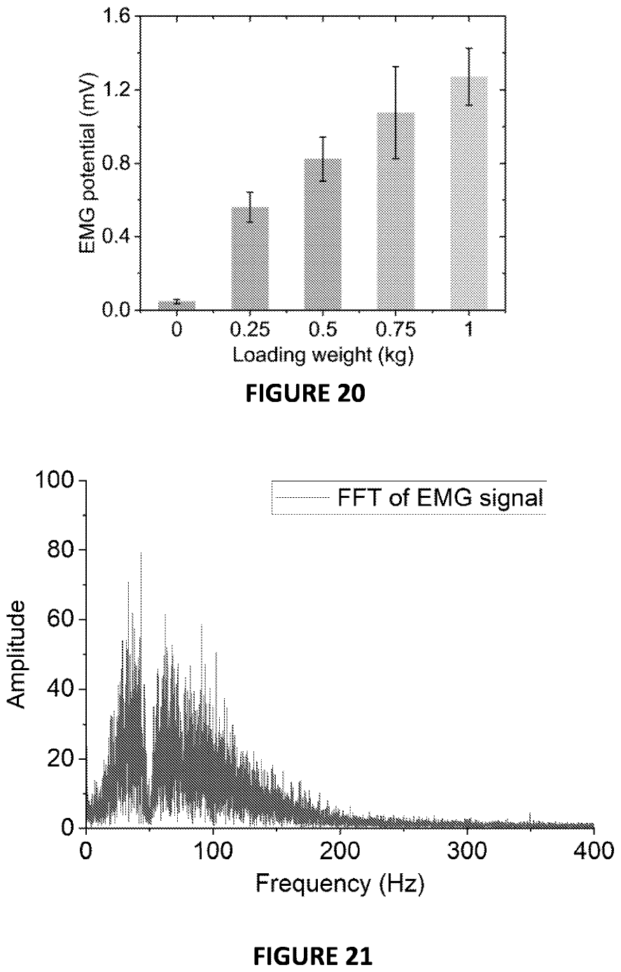

A fibre sensing element 75 weighing approximately 10 μg deposited around the thumb-thenar region 7 for EMG measurement is shown in FIG. 18. An array of 360 fibres were deposited over a length of approximately 2 cm, and during deposition the apparatus 10 moved in a linear manner at a constant speed of 50 μm/s to allow fibre deposition covering the muscle area. Electrodes 80 are connected to the sensing element 75 for EMG data collection. A load tied to the thumb exerts a force (F) on the thumb which is counterbalanced by contraction of the muscles in the thumb-thenar region 7. EMG signals acquired by the fibre sensing element 75 are shown in FIG. 19 and illustrate the sensitivity of the present sensing element 75 during periods of muscle contraction and relaxation. FIG. 20 shows the different mean EMG potentials with increasing weight. FIG. 21 shows the frequency response of the captured EMG data falling within standard frequency ranges of muscle response, further indicating the utility of the present fibres 70. After completing the data capture, the fibre sensing element 75 can be wiped off.

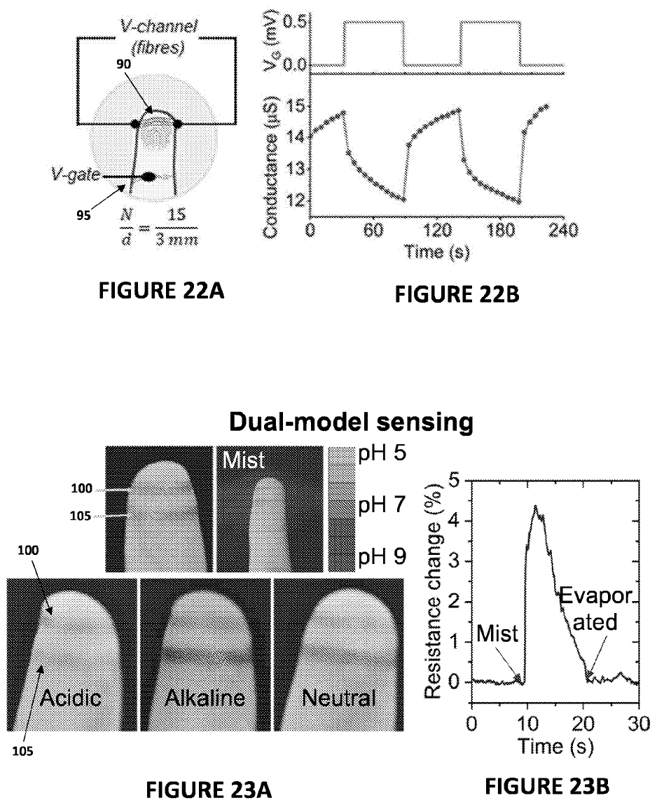

Due to the semi-conducting nature of PEDOT:PSS, the electrically-conductive fibres 70 can be configured as a skin-gated organic electrochemical transistor (OECT) as shown in FIG. 22A. The skin-gated OECT includes a fibre channel 90 and a gate 95 and shows typical depletion-mode operation polarity (see FIG. 22B), which is consistent with other OECTs using PEDOT:PSS as their primary channel material. For a typical deposition of 2 mins (i.e., 120 fibre loops on skin), the OECT response time was estimated to be in the range of minutes. Thus, this imperceptible skin-gated OECT holds promises for future clinical applications in measuring quasi-static continuous biological signals with long fluctuation time in hours, such as for circadian rhythm and epilepsy monitoring.

Different fibres configured to sense different properties can be used to provide multi-modal sensing elements 75 at the same location on the target. FIG. 23A shows electrically-conductive fibres 100 and colour-responsive fibres 105 both looped on the same index finger. The pH-responsive fibres 105 were able to distinguish between impinging vinegar (acidic), water (neural), and laundry powder (alkaline) mist flows which were passed over the finger by simultaneously monitoring the electrically-conductive fibres' resistance change (see FIG. 23B), and the pH-responsive fibres' colour change (see FIG. 23A). It is also to incorporate other biochemical recognition molecules, coupled with suitable signal transduction modalities in different fibres. This could provide wearable sensors-for-plants in probing intricate communications and interactions between microbes/insects and plants.

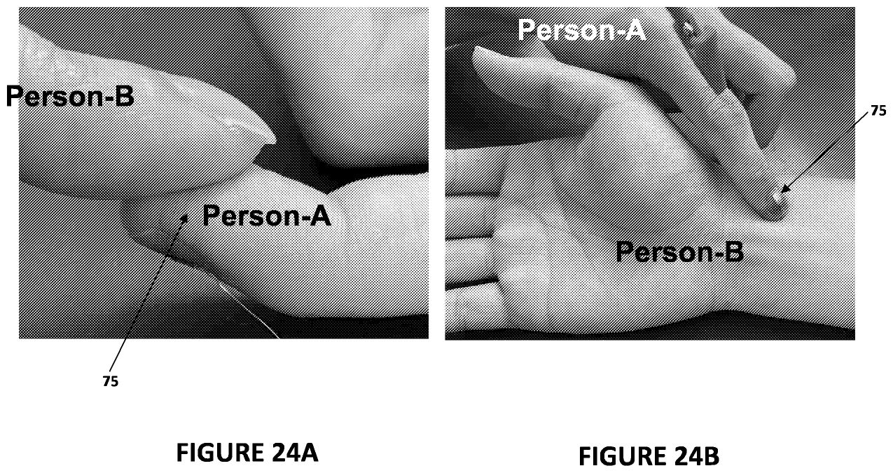

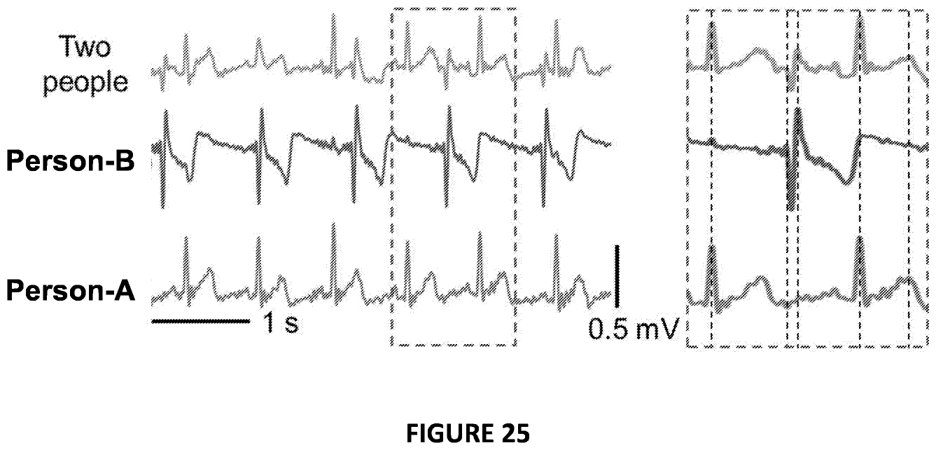

The present fibres also allow for unconventional sensing functionalities. As both sides of the electrically-conductive fibres are exposed when worn on the finger, a wearer (Person A) can detect another individual's (Person B) ECG by contacting the wearable electrode 75 against the Person B's bare finger or wrist (see FIGS. 24A, 24B). The top organic electrochemical transistor (OECT) chart of FIG. 25 illustrates the augmented ECG data captured through the fibre array, whereas the middle and lower charts show the ECG data for Person A and Person B individually captured at the same time for reference. In this example, 180 fibres were deposited over a length of 3 mm onto the index fingers of Person A.

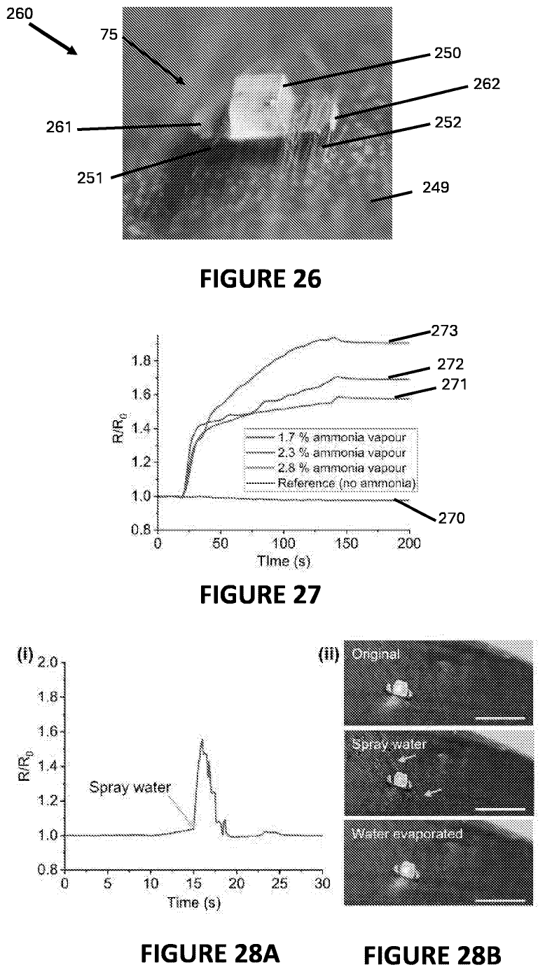

FIG. 26 shows an exemplary application of the apparatus of FIG. 1. In this example, the apparatus of FIG. 1 has been used to produce a sensing element 75, operable to measure vapour present in the environment surrounding the sensing element 75.

In this example, the fibres have been deposited onto an orchid leaf 249. As shown in FIG. 26. The fibres physically bind an electronics component 250 to the orchid leaf 249. The sensing element 75 and the electronics component 250 form part of a sensor 260 for detecting the presence of vapour or liquid on or surrounding the orchid leaf 249.

In this example, the electronics component is an LED. The sensing element 75 comprises a first array of fibres 251, which contact a first electrical contact 261 of the LED, and a second array of fibres 252 which contact a second electrical contact 262 of the LED.

FIG. 27 illustrates a plot of the normalised resistance against time for tests where ammonia vapour was applied to the orchid leaf 249 at 20 s and stopped at 150 s. Line 271 illustrates the results of a first test wherein the concentration of ammonia vapour was 1.7%. Line 272 illustrates the results of a second test wherein the concentration of ammonia vapour was 2.3%. Line 273 illustrates the results of a third test wherein the concentration of ammonia vapour was 2.8%. Line 270 illustrates the results of a control experiment wherein no ammonia vapour was applied to the orchid leaf 249.

As shown, the measured resistance of the sensor 260 increases irreversibly as the concentration of ammonia vapour increases. In this way, the recorded resistance may be used to not only detect the presence of ammonia vapour but also determine the concentration of ammonia vapour present.

FIG. 28A illustrates a plot of the normalised resistance of the sensor 260 against time for tests where water droplets were sprayed onto the orchid leaf 249 at 15 s. As shown, the resistance increases when the water droplets are applied to the orchid leaf 249 but returns to a baseline resistance after a period of evaporation.

FIG. 28B shows the sensor 260 prior to the water spray (top), immediately after the water spray was applied to the orchid leaf 249 (middle), and after the water had evaporated from the orchid leaf 249 (bottom). As shown, the LED is illuminated when the orchid leaf 249 is dry and not as brightly illuminated when the orchid leaf 249 is wet. In this way, the LED itself may act as a visual indicator of the presence of detected water droplets. Such an indicator may be used in crop management systems.

It will be understood that FIGS. 26 to 28B illustrate examples of sensing applications and that the sensor 260 may be operable to detect other liquids and/or other vapours such as CO₂.

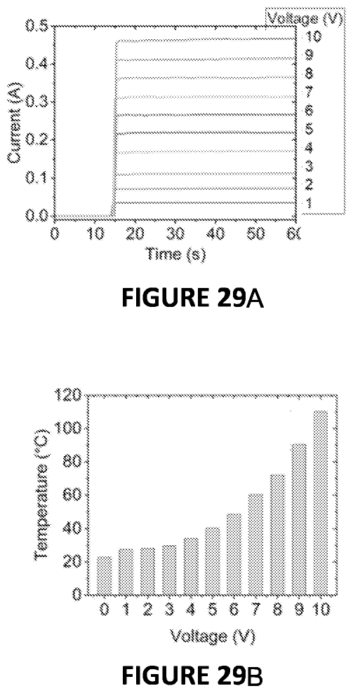

FIGS. 29A and 29B show data collected from an exemplary application of the apparatus of FIG. 1. In this example, the apparatus of FIG. 1 has been used to produce a device for heat generation. The device comprises an array of fibres that have been deposited onto a freestanding object. In this example, the freestanding object is a glass slide. The liquid composition for the fibre array comprises nanoparticles. In this example, silver nanoparticles (AgNP) were used as the electrically-conductive component.

Around 2000 parallel fibres were deposited onto the glass slide to form the fibre array. Both ends of the fibre array were connected to a power supply (IPS 2303S, RS PRO) and connected in series with a multimeter (34465A, Keysight Technologies).

FIG. 29B shows a current-voltage relationship for various supply voltages from 1V to 10V. FIG. 29C shows temperature measurements recorded using an IR-camera (Teledyne FLIR) at a center of the array for the supply voltages from 1V to 10V. As shown, the fibre array increased the temperature from temperature (21° C.) to around 110° C. with 10 V voltage input. This correlates to ~4.6 W heating power with 10 V voltage input.

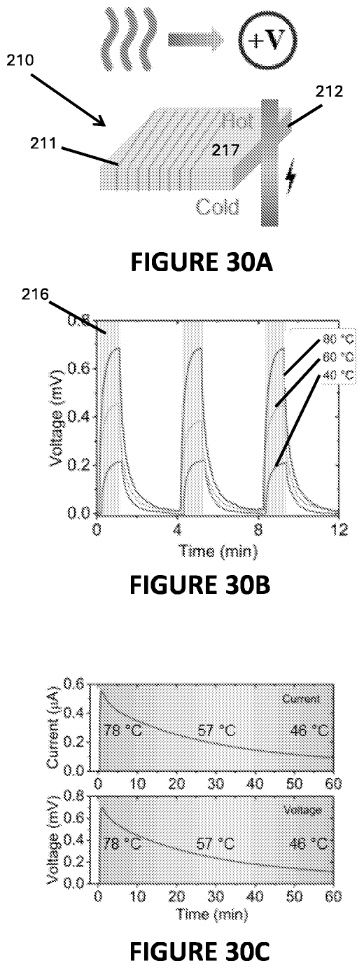

FIG. 30A shows an exemplary application of the apparatus of FIG. 1. In this example, the apparatus of FIG. 1 has been used to produce a thermoelectric device 210. The thermoelectric device 210 is operable as a temperature sensor or as an energy harvesting device. For example, the thermoelectric device could generate a voltage upon application of a temperature difference. The thermoelectric device 210 comprises a fibre array 211 that has been deposited onto a freestanding object. In this example, the freestanding object is a coaster 212 for resting drinks thereon. The liquid composition for the fibre array comprises a thermoelectric material as the functional component. In this example, PEDOT:PSS was used as the functional component.

The fibres of the fibre array 211 circumscribe the coaster 212. In this example the fibre array 211 had a length of 40 mm and a width of 100 mm. FIG. 30B shows the voltage generated from the device 210 when a hot plate (not shown) of controlled temperatures was placed onto a top surface 217 of the coaster 212. Shaded areas 216 indicate periods of time where the hot plate rested on the top surface 217. A multimeter (34465A, Keysight Technologies) was used to measure current and voltage output. As shown, the measured voltage of the device 210 increases with applied temperature and exponentially decays when the heat source is removed.

This effect is further highlighted by FIG. 30C which illustrates the current and voltage response of the device 210 when a bowl of 80° C. water was placed on the top surface 217 and allowed to cool. FIG. 30C indicates that power is generated by the thermoelectric effect. The device 210 may be used for energy harvesting or temperature sensing from a variety of heat sources in the home or workplaces.

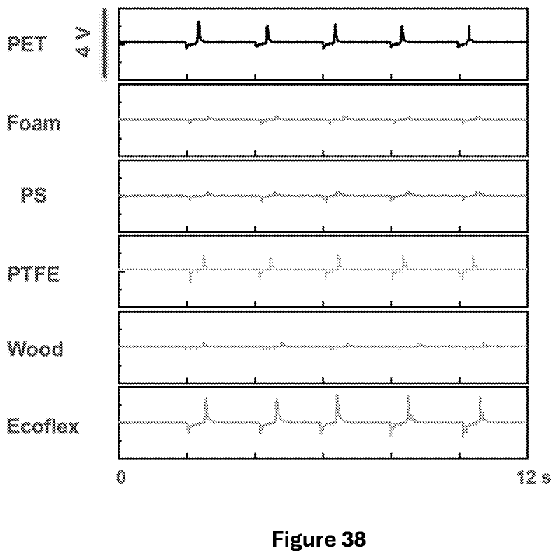

FIG. 38 shows data collected from an exemplary application of the apparatus of FIG. 1. In this example, the apparatus of FIG. 1 has been used to produce a sensing element operable to characterise surface materials. In particular, the sensing element may characterise the surfaces by triboelectrically generated charges.

In this example, bio-electric fibres have been deposited onto a human hand and electrically connected to a voltmeter (not shown) arranged to measure a voltage output of the fibres. FIG. 38 illustrates results of surface impact experiments performed using such a sensing element. A variety of different materials were tested including Polyethylene terephthalate (PET), foam, Polytetrafluoroethylene (PTFE), Wood and Ecoflex. During testing the fibres of the sensing element were periodically tapped against a surface of the various materials and an output voltage of the sensing element was recorded. As shown in FIG. 38, the signal relative peak-to-peak signal strength and signal patterns are material-dependent. As such, the fibre array may be used to characterise the composition of a surface which it contacts.

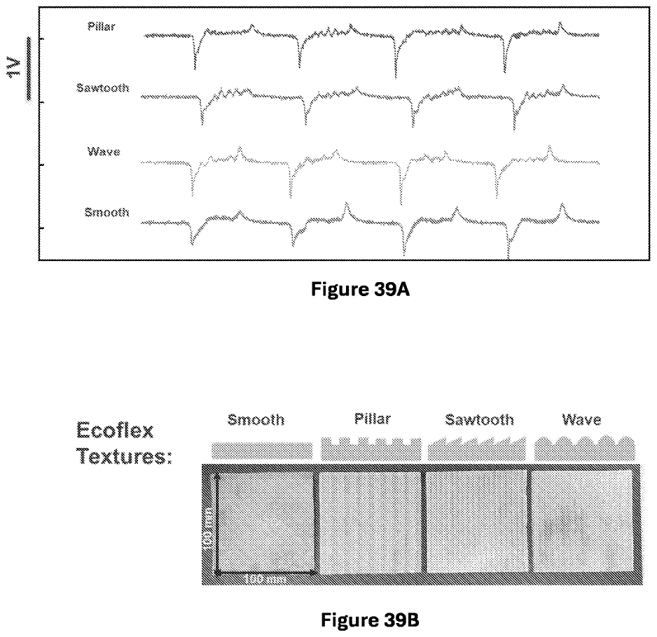

FIG. 39A shows data collected from an exemplary application of the apparatus of FIG. 1. In this example, the apparatus of FIG. 1 has been used to produce a sensing element operable to characterise surface textures. In particular, the sensing element may characterise the surfaces by their piezoelectrically generated charges.

In this example, bio-electric fibres have been deposited onto a human hand and electrically connected to a voltmeter (not shown) arranged to measure a voltage output of the fibres. FIG. 39B illustrates four different surface textures that were tested during surface contact experiments performed using such a sensing element. As shown, a variety of different surface textures were tested including a smooth surface as well as surfaces defined by: a wave profile, a square wave profile (pillar), and a sawtooth profile. Each of the surfaces tested consisted of the same material. In this example, each of the surfaces tested was formed from a synthetic rubber (Ecoflex).

FIG. 39A illustrates results of surface contact experiments performed on the surfaces of FIG. 39B. During testing the fibres of the sensing element were slid across the surface being tested and an output voltage of the sensing element was recorded. As shown in FIG. 39A, different surface textures produce electrical (voltage) signals, and the patterns of the electrical signals are dependent on the texture type of surface tested. As such, the fibre array may be used to characterise the texture of a surface which it contacts.

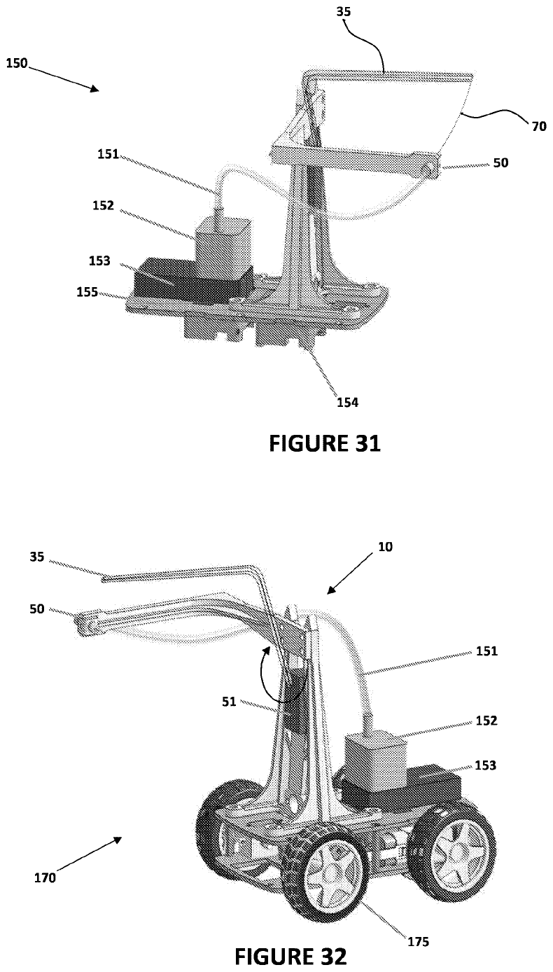

FIG. 31 illustrates a hand-held apparatus 150 for forming fibres 70 around a target object 3. As the user is able to manually orient the apparatus 150 in the desired orientation to provide the desired fibre pattern, it is not necessary to include the coupling 20 described above. However, in some cases, the coupling 20 may be included in apparatus 150 to provide additional degrees of freedom, such that the user merely holds the apparatus 150 close to the target 3 and the additional degrees of freedom provide greater flexibility in the application of fibres. This is particularly advantageous where access to the target 3 is limited. The apparatus 150 includes tubing 151 to deliver the fibre-forming liquid composition from a reservoir containing a pump 152, for example a piezo air pump and a battery 153 for powering the apparatus 150. As the rotating arm 35 moves from the dispenser 50, for example a syringe, the fibres 70 are drawn in the manner described herein. The apparatus 150 is also shown mounted to an acrylic plate having a controller 154 mounted thereto for controlling the operation of the components described herein. An Arduino is an example of a suitable controller. The acrylic plate has one or more gripping regions 155 where a user can comfortably hold the apparatus 150, for example when moving the apparatus 150 around the target object.

FIG. 32 illustrates an apparatus 170 in the form of a robotic vehicle having at least one driven wheels 175 and an apparatus 10 mounted thereto. The robotic vehicle can be controlled remotely by a user which allows for remote fibre deposition onto a target. The robotic vehicle includes a reservoir of fibre-forming liquid composition, fluid lines to fluidly connect the dispenser 50 to the reservoir, and a battery-powered micropump to pump fibre-forming liquid composition through the dispenser 50. The robotic vehicle can periodically shift its position as fibres 70 based on the position of the arm 35 to provide an array of cellulose-based sensing fibres 70, for example using a controller on board the robotic vehicle which is operatively coupled to the motor driving the driven wheels 175 and the motor 51 driving the arm 35.

The apparatus 10 is able to draw sensing fibres 70 from the fibre-forming liquid composition 65 at much lower speeds compared to in situ spinning devices, for example, between 45-65 rpm, which results in much wetter sensing fibres 70 being drawn compared to those drawn in in situ spinning devices, which operate at a higher speed and therefore dry quicker due to the higher surface area to volume ratio of each drawn fibre. A further advantage of operating at relatively low speeds is the present apparatus 10 is able to deposit fibres in a controlled manner at much lower fibre densities (e.g., approximately one fibre every 1-50 μm, for example 20 μm) compared to in situ spinning devices which deposit fibres in a much higher density, resulting in the host perceiving the deposited fibre device. Controlling the fibre number density allows tuning of the bulk optical property of the fibre patterns from semi-transparent (transmittance approximately 90%) to visible (transmittance approximately 80%) for the colour-responsive fibres (e.g., pH-responsive fibres) used for visual colorimetric readouts. Fibres with various orientations can also be created using the apparatus 10 as explained herein. Each rotating arm orbit results in one strand of solution fibre being deposited on the target 3 and spontaneously morphing to the target surface. The low bending stiffness of the micro-scaled fibre allows such tethering to take place across biological objects of various sizes, from the width of a human hair.

The fibre deposition apparatus 10 utilises orbital spinning to produce fibres. However, it will be understood that the movement of the arm 35 from the first position to the second position needn't be limited to rotational movement. For example, the actuator may be operable to linearly translate the arm between the first position and the second position.

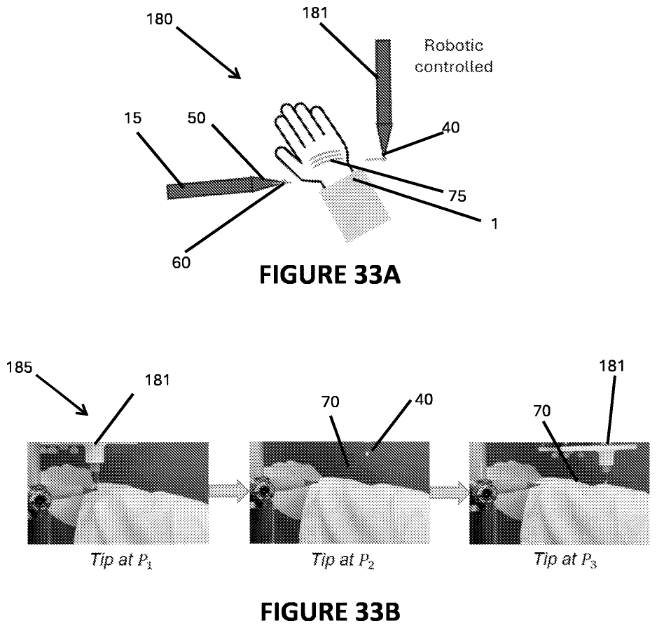

FIG. 33A illustrates a fibre deposition apparatus 180 for depositing fibres 70 according to the present method. References have been used for corresponding features of the apparatus 10.

The apparatus 180 comprises a movable arm 181 and a support frame 15. A dispenser 50 is mounted to the support frame 15. In this example, the dispenser 50 is mounted at an apex of the support frame 15. The apparatus further comprises a motor (not shown) operable to translate the robotic arm 181 with respect to the support frame 15. In this example, the movable arm 181 comprises a multi-axis robotic arm operable to perform 3D translation. The apparatus 180 further comprises a controller (not shown) for actuating a tip 40 of the movable arm 15 within a deposition zone (not shown).

The dispenser 50 dispenses the fibre-forming liquid composition in a controlled manner at an outlet port 60. The outlet port 60 is at an apex of the dispenser 50 and the fibre-forming liquid composition is continuously supplied to the dispenser 50 such that the fibre-forming liquid accumulates on the outlet port 60.

FIG. 33B illustrates an exemplary method 185 for deposition of fibres 70 onto a freestanding object 1 using the apparatus 180. In this example, the robotic arm used was SWIFTI CRB 1100, ABB and its controller was setup following the ABB official instruction.

As shown, during deposition the tip 40 is moved from a first position (P1) to a second position (P2) to a third position (P3). At P1, the tip 40 contacts the fibre-forming liquid composition accumulated on the outlet port 60 (left image of FIG. 33B). From P1 to P2, the tip 40 moves away from the outlet 60 and the fibre-forming liquid composition is mechanically drawn away from the outlet 60 into a fibre 70. From P2 to P3, the tip 40 moves such that the fibre 70, between the outlet 60 and the tip 40, contacts the freestanding object 1 depositing the fibre 70 thereon. The deposition of fibres 70 is seemingly imperceptible to the object 1, which may be a living object such as a plant or person.

The controller may be programmed to repeat the stages of method 185 with varied P2 and P3 coordinates to form a fibre array 75 as shown in FIG. 33A. It will be appreciated that the coordinates of P3 and P3 will depend on the location and dimensions of the fibre array 75 to be produced.

In this example, the fibre 70 is drawn as the tip moves from P1 to P2 and the fibre is placed on the freestanding object when the tip moves from P2 to P3. In other examples, the tip 40 may be moved in a single continuous action to draw and deposit the fibre 70.

As shown in in FIG. 33B, the moveable arm 181 can achieve 3D transformations of the tip 40. As the movable arm 181 has a greater degree of freedom than the arm 35, the apparatus 180 may be able to more easily deposit complex fibre arrays than the apparatus 10. Such an apparatus 180 is particularly suited for applications which require discontinuous fibre arrays.

By operating at relatively low speeds, the present apparatus 10 is able to draw fibres 70 in a wet, sol-gel state which remain wet when they contact the target 3. As the wet sensing fibre 70 dries on the surface of the target 3, it adheres to the surface of the target 3. The level of conformity between the deposited fibre 70 and the surface of the target 3 can be controlled by the composition of the fibre-forming liquid composition 65 as discussed below.

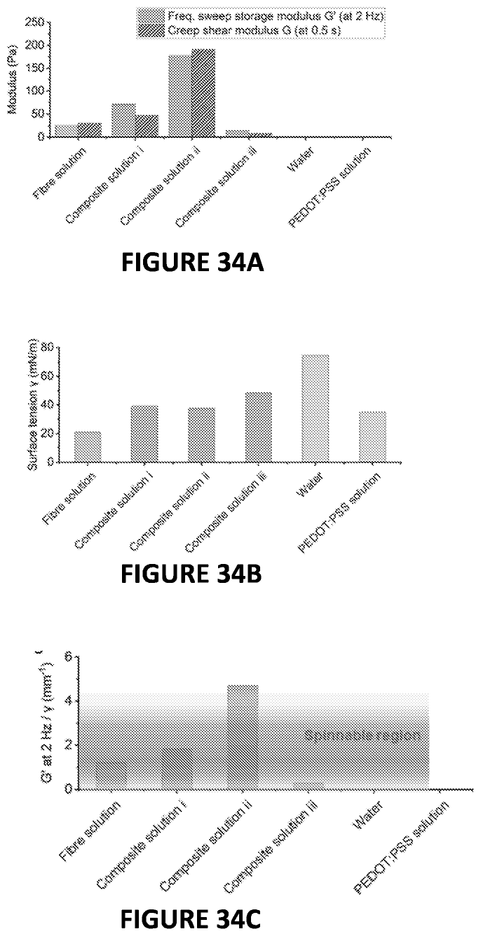

In an example, to produce feedstock fibre-forming liquid compositions 65, polyethylene oxide (PEO) was used as the fibre-forming polymer in the fibre-forming liquid composition. Mixtures of functional materials were then dispersed in water or a water/acetone co-solvent with PEO for dispensing. Rheological characterisation using shear creep test confirms that all the solution formulations are in the percolated states. During the orbital fibre spinning, each fibre is initially drawn from the nozzle tip through a sudden input of mechanical shearing, and the fibre is subsequently extended by the rotating arm 35. Molecular chains within in the solution exist in a long-range, weakly percolated state which allows the solution to be drawn into a fibre extending between the nozzle tip and the rotating arm prior to the fibre's surface tethering. An exemplary parameter for identifying suitable fibre-forming liquid compositions is the elastic storage modulus (G′) of the solution. The countering effect for fibre formation is the solution surface tension (γ). If the surface tension is too high, then the fibres may have beads-on-string structures or the fibre may break away from the solution. The ratio of G′ to γ may be used as an indicator to characterise the solution ‘spinnability’ under this technique. This is illustrated by FIGS. 34A to 34C which show the elastic storage modulus (G′), surface tension (γ) and ratio of G′ to γ of the fibre solution and alternative composite solutions i-iii.

The elastic storage modulus G′ was determined via shear creep rheological experiment under parallel plate configurations with a Kinexus KNX2112 machine, in which the input shear stress was 1 Pa. Each composite solution was dissolved in water. Composite solution i consisted of PEO 8M Da, 2% w/w. Composite solution ii consisted of PEO 8M Da, 2% w/w and +HA 0.5% w/w. Composite solution iii consisted of and PEO 4M Da, 2% w/w. The defined creep shear modulus (G) was recorded at 2 Hz while the storage modulus (G′) values were recorded at time point 0.5s. 0.5s corresponds to the time from fibre initiation to deposition on the target object. The parameters for recording the shear modulus (G) and the storage modulus (G′) may be selected to optimise the measurement technique for specific apparatus geometries.

Incorporating hyaluronic acid (HA) into long-chain polyethylene oxide (PEO) could be useful in promoting a robust chain percolation as indicated by increased shear modulus.

However, excessively strong molecular chain connection may make a solution un-drawable due to elastic re-coil and hindered chain sliding. This places an upper limit on a region of ‘spinnability’ in the ratio of G′ to γ. As shown in FIG. 34C, the fibre solution and composition i fall within the spinnable range and are therefore suitable for use in the present apparatus.

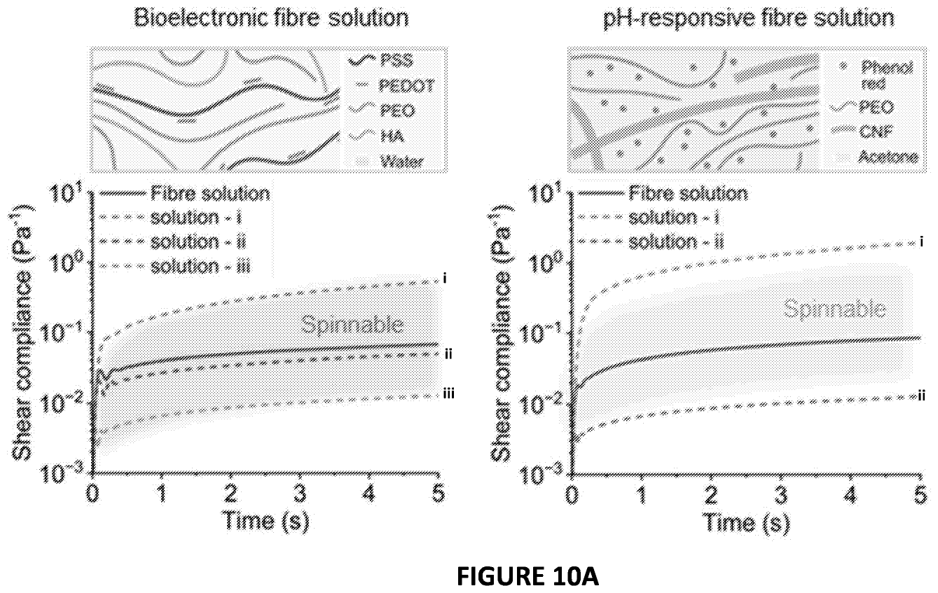

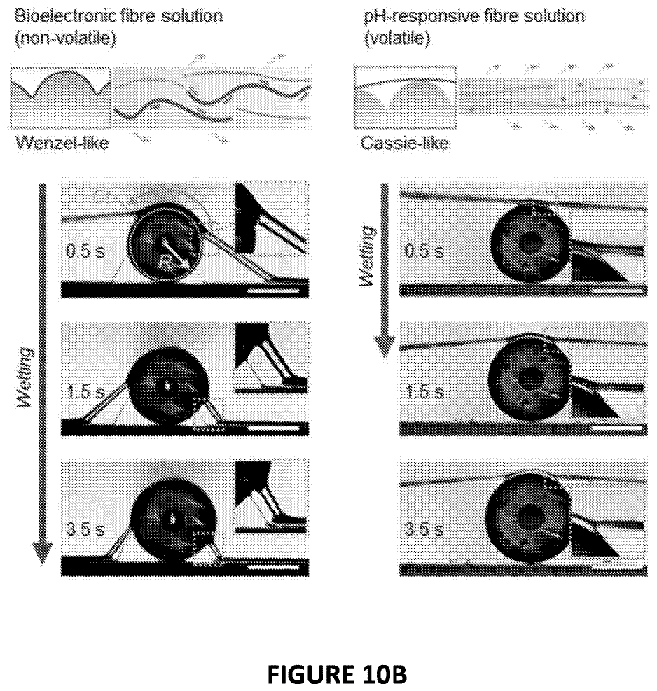

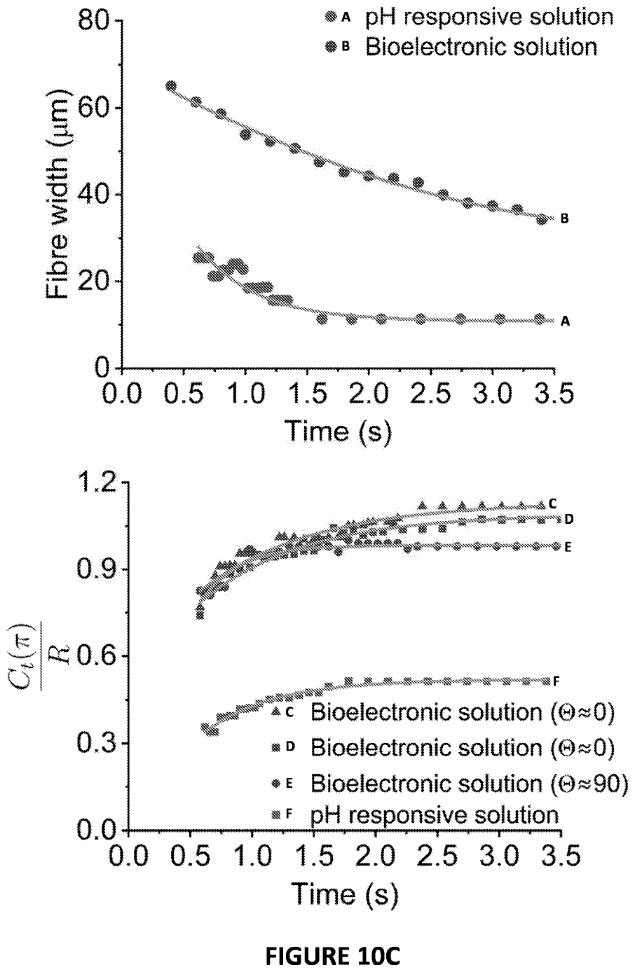

Another parameter for identifying suitable fibre-forming liquid compositions is a shear creep test which characterises the solution's deformation (shear strain) under a step shear stress input. FIG. 10A illustrates the shear compliance of electrically-conductive fibres and pH-responsive fibres following a 1 Pa input stress at 25° C. The results show the time-dependent shear compliance (inverse of shear modulus) for comparative formulations of the electrically-conductive fibre-forming liquid composition, and the pH responsive fibre-forming liquid composition. Despite there being distinct base solvents used for the two formulations (aqueous versus organic), a shear compliance of between approximately 10⁻² to 10⁰ Pa⁻¹ was found to be suitable for use in the present application. Incorporating cellulose nanofibers (CNF) or hyaluronic acid (HA) into long-chain polyethylene oxide (PEO) was also useful in promoting robust chain percolation, as indicated by decreased shear compliance. However, as mentioned previously, strong molecular chain connection would also make the solution unsuitable, likely due to elastic re-coil which would hinder chain sliding.

One exemplary solution for forming electrically-conductive fibres is a mixture of PEDOT:PSS solution (an example of an electrically-conductive component) and PEO solution. The reference solutions referred to in FIG. 10A are made up of PEO (8M Da), HA and PEDOT:PSS. Specifically, reference solution i) to iii) (shown as the upper, middle and lower lines in the left graph of FIG. 10A) are PEO (4M Da), PEO (8M Da) and PEO (8M Da) and HA respectively. The pH-responsive fibre solutions are PEO (8M Da) and 3% (w/w) CNF and phenol red. Specifically, reference solution i) (shown as the upper line in the right graph of FIG. 10A) reduces the CNF concentration to 1% (w/w) and reference solution ii) (shown as the lower line in the right graph of FIG. 10A) is measured in an open-air condition when the solvent in the normal pH-responsive fibre solution is allowed to evaporate.

It has also been found that the level of fibre “wetness” (i.e., the level of residual solvent upon contact) determines whether a fibre tethers in a dominantly Wenzel-like state or a Cassie-like state (with reference to FIGS. 15 and 16 described above showing conforming 70 and non-conforming 72 fibres). Evaluating fibre diameter changes over time, as shown in FIG. 10B, indicates that for the pH-responsive fibre formulation, the majority of the solvents are evaporated during the fibre initiation and drawing stage. As a result, a Cassie-like contact state is resulted upon fibre tethering (from 0.5 s onwards). For the electrically-conductive fibre formulation (water based), abundant residual water remains in the ‘wet fibre’ upon surface tethering, thus a dominant Wenzel-like state is resulted (see lower images in FIG. 10B). FIG. 10B shows the time dependent wetting process for an electrically-conductive fibre (left image), and a pH-responsive fibre (right image) (scale bars, 500 μm). Magnified left images compare an electrically-conductive undergoing continuous wetting for approximately 3.5 s after in-air spinning, and a previously deposited dried fibre with much thinner diameter. Magnified right images shows that the pH-response fibre completes surface wetting within the first 1.5 s after in-air spinning.

FIG. 10C upper image shows fibre widths versus time upon fibre tethering on a glass surface for the two solutions. FIG. 10C lower image shows Plots of fibre solutions' normalised contact line length (Cₜ(π)/R) versus time for the two solutions. For a substrate feature of hundreds of microns in diameter (i.e., representing topographical features of finger ridges and orchid flower petals), differences in surface hydrophobicity only have minor effects on the relative contact line coverage likely due to the amphiphilic nature of PEO. For the electrically-conductive fibre-forming liquid composition, treated glass surfaces with various hydrophilicities are tested. Here, Cₜ is the time-dependent contact line length, and R is the radius of the curved glass surface and θ indicates the water contact angle for the treated glass surface. It is understood that when comparing the driving capillary force for spreading of a wet fibre versus the resisting elastic force, it is the driving capillary force that will dominate. Hence, for the electrically-conductive fibre formulation, intimate contact over hundreds of microns of topographical features are expected to form on convex and solid structures.

The exemplified fibre-forming liquid compositions have less than 3% solid material (e.g., between 2 and 2.5%) in water. The term “solid material” includes PEO, for example where the PEO used was 8 Mda in molecular weight. Consequently, it may be necessary to evaporate 98% of water as the deposited fibres dry and adhere to the surface of the target. The increased fibre “wetness” leads to more contributions from surface tension to form conformal contact. While such a “wet” fibre can make it more difficult to draw a continuous fibre using conventional methods, such as in situ-spinning, the present methods and apparatus allow for fibres to be drawn from such solutions, thus achieving the improved functionality (e.g., specific fibre patterns, increased utility, such as conductive or pH-responsive devices) described herein.

Typically, when the solvent in the fibre-forming liquid composition comprises water the fibre-forming liquid compositions have been found to provide conformal fibre attachment (akin to the Wenzel state), exemplified by experiments on the surfaces of an orchid flower petal (bare surface water contact angle of approximately) 140° and a leaf (bare surface water contact angle) 80°. Alternatively, when the solvent in the fibre-forming liquid composition comprises water and an organic solvent (e.g., acetone), this was found to resulting suspended fibres (akin to the Cassie state) on the same surfaces.

For all fibre 70 types, a feature width (e.g., a fibre diameter) of 2 μm to 20 μm (e.g. 3 μm to 20 μm) can be achieved. In some cases, the diameter can be between 5 μm to 20 μm. For electrically-conductive PEDOT:PSS fibres, a conductivity of 10⁴ S/m can be achieved. For electrically-conductive AgNP fibres, a conductivity of ~1×10⁵ S/m can be achieved.

In the examples described above, the electrically-conductive fibres could be removed from the target objects after use and be recycled into new conducting 3D printing fibre-forming liquid compositions. As an example, the recycled fibres, in a dry state, could be redispersed into 70% ethanol (advantageously acting as a sterilisation medium) at a concentration of 1.5% (w/w). 1% of 400 k Da PEO powders can also be added. The dispersion can then be ground for 5 minutes to form a homogeneous solution paste, which can be loaded into a syringe for dispensing.

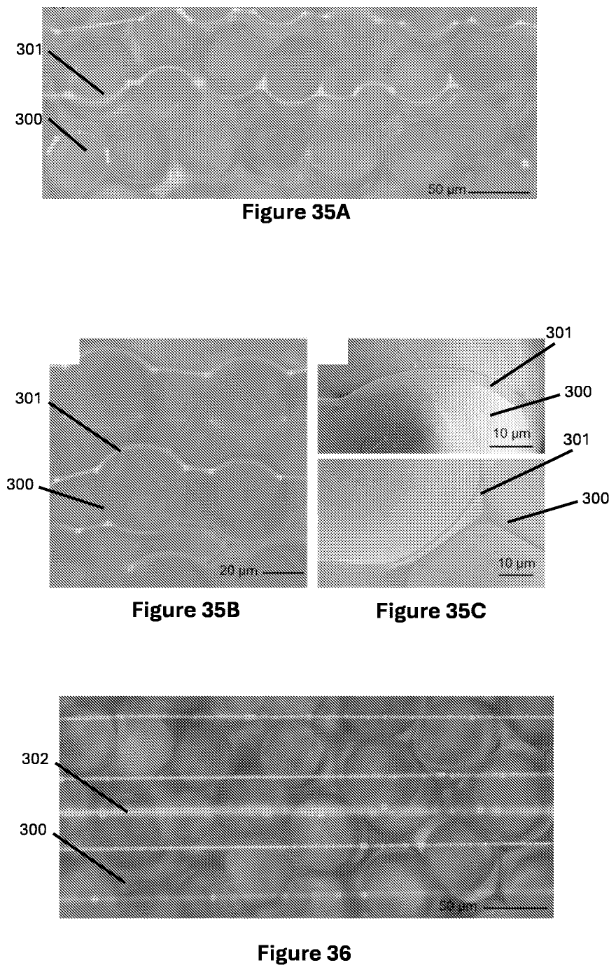

FIGS. 35A-36 illustrate examples of an object 300 with a plurality of fibres deposited thereon. In each example, the fibres were deposited using the fibre deposition apparatus 10 described herein. In each example, the object 300 is an orchid flower petal. FIGS. 35A-35C illustrate a first example wherein electrically-conductive (bioelectronic) fibres 301 were deposited on the object 300. FIG. 35A-35B illustrate microscopic images of the object 300. FIG. 35C illustrates two SEM images of the object 300. In this example, PEDOT:PSS was used as the functional component in the electrically-conductive fibres 301. The fibres 301 were doped with a fluorescent dye (Fluorescein Sodium (20 μg/mL, Sigma-Aldrich)) for improved visibility in the microscopic images. As shown, the electrically-conductive fibres 301 are morphed to the surface topography of the petal. In this example, the fibres 301 are in contact with the surface topography of the object 300 along over 50% of the length of the fibres 301.

FIG. 36 shows a microscopic image of further example wherein a plurality of PEO-based fibres 302 were deposited on the object 300. In this example, the PEO was prepared using a volatile solvent (2% 8 M Da PEO dissolved in 80% v/v ethanol). As shown in FIG. 36, the PEO fibres have not morphed to the surface topography of the object 300. Instead the fibres 302 are suspended on top of the surface of the object 300.

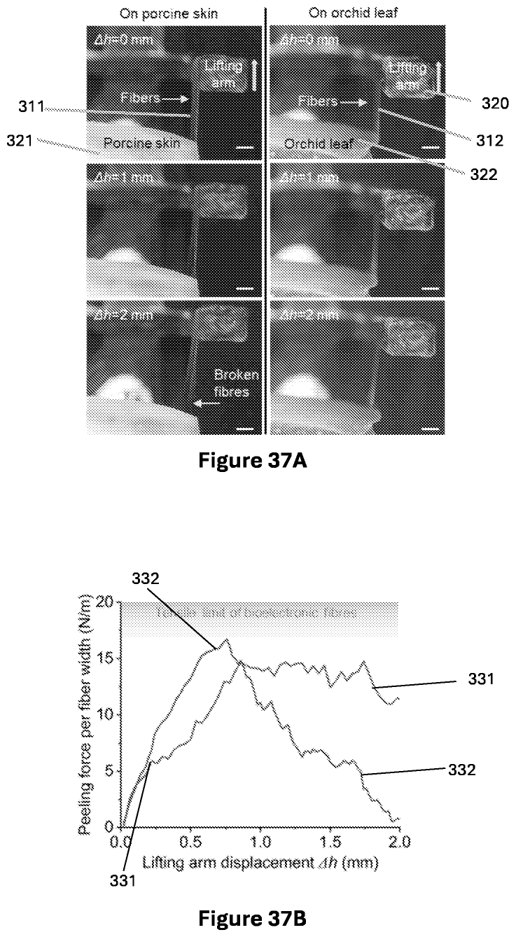

FIG. 37A illustrates a 90-degree peeling test performed on: an array of electrically-conducting fibres 311 deposited onto a porcine skin substrate 321 (left of FIG. 37A); and an array of electrically-conducting fibres 312 deposited onto an orchid leaf substrate 322 (right of FIG. 37A). Each of the fibre arrays 311, 312 were linear arrays (evenly spaced parallel lines) deposited using the fibre deposition apparatus 10. In particular, the fibres 311, 312 of each array were oriented perpendicular to the lifting arm 320. In this example, each of the arrays of fibres 311, 312 comprised around 200 fibres deposited over a width of 10 mm. Scale bars located in the bottom right of each image in FIG. 37A represent 2 mm.

In this example, the peeling test was derived from the ASTM D2861 90-degree peeling test. As shown, the arrays of fibres 311,312 may be deposited onto their respective substrate 321, 322 and onto a lifting arm 320. During testing, the lifting arm 320 is raised and a peeling force the fibres 311,312 exert on the lifting arm 320 recorded in Newtons.

FIG. 37B illustrates surface adhesion characterisations for the bioelectronic fibre arrays 311, 312 tested using the 90-degree peeling test of FIG. 37A under dry conditions. Peeling force per unit fibre width was estimated by:

$$\frac{F}{3 \times 1 {0}^{- 6} \cdot N} ,$$

where F is the peeling force in Newtons, N is the number of fibres deposited, and 3×10⁻⁶ m is the assumed mean width of each fibre. In FIG. 37B, peeling force per unit fibre is plotted against lifting arm height for each of the two fibre arrays 311,312 which correspond to line 331 and line 332 respectively.

As shown, the maximum recorded peeling force per fibre width is ~15 N/m for both fibre arrays 311, 312. This may be a result of the extensive fractional length of the fibres 311, 312 which contact the substrates 321, 322 (i.e., high topographical conformity) and the state of the fibres when they are deposited on the substrates 321, 322. This is highlighted by FIG. 37A which illustrates that the peeling process ultimately resulted fibre breakage instead of noticeable delamination.

### Liquid Composition

According to a further aspect of the present disclosure, there is provided a liquid composition for forming a drawn fibre. The liquid composition comprises a fibre-forming polymer; a solvent; and a functional component. The liquid composition is also herein referred to as a ‘fibre-forming liquid composition’ as described in the method of depositing a fibre onto a freestanding object using a fibre deposition apparatus described herein. In other words, the liquid composition can be the fibre-forming liquid composition as described in a method of depositing a fibre onto a freestanding object using a fibre deposition apparatus as described herein, a fibre deposition apparatus for depositing fibres onto a freestanding object as described herein, and/or a sensor as described herein. Typically, the liquid composition is suitable for forming drawn fibres suitable for use in sensing elements (e.g., for bioelectronic sensing and/or pH sensing).

As used herein, the term “fibre-forming polymer” refers to any suitable polymer capable of forming a fibre. Generally, a fibre-forming polymer is capable of acting as a base to facilitate fibre drawing, for example, in a method of depositing a fibre onto a freestanding object using a fibre deposition apparatus as described herein.

Any suitable fibre-forming polymer may be used in the composition of the present disclosure. For example, the fibre-forming polymer may include one or more polymers selected from the group consisting of polyethers, polycarbonates, polyamides, polyimides, polyolefins, halogenated polymers, aromatic polymers, ketone polymers, methacrylate polymers, polyesters, cellulose derivatives, polypeptides, polysulfones, polyurethanes, polyureas, and glycosaminoglycans, polypeptides, polysaccharides, polyphenols, proteoglycans and polynucleotides. In some examples, the fibre-forming polymer comprises a polyether and/or an aromatic polymer.

Preferably, the polyether is polyethylene oxide (PEO). The fibre-forming polymer may comprise, or consist essentially of, PEO. In some examples, the fibre-forming polymer comprises a polyether and/or a glycosaminoglycan (for example, the fibre-forming polymer may comprise PEO and/or hyaluronic acid (HA)).

The fibre-forming polymer may comprise, or consist essentially of, a glycosaminoglycan (e.g., HA)). Generally, the presence of glycosaminoglycans (e.g., HA) in the composition may decrease the overall shear compliance of the composition (e.g. resulting in an improvement in the spinnability of the composition). In some examples, the fibre-forming polymer comprises PEO and HA. The weight ratio between PEO and HA may be between around 1 to 10:1, preferably between around 1 to 7:1, more preferably between around 2 to 6:1, even more preferably between around 2 to 4:1. In some examples, the weight ratio between PEO and HA is between around 3.5 to 4.5:1, e.g., 4:1.

Preferably, the aromatic polymer comprises one or more phenyl rings. More preferably, the aromatic polymer is polystyrene. The fibre-forming polymer may comprise, or consist essentially of, polystyrene.

In principle, a suitable solvent can be selected depending on the fibre-forming polymer in the liquid composition. The role of the solvent is to solubilise the polymer such that the liquid composition is in a percolated state. Typically, the molecular chains of the fibre-forming polymer forms a long-range, weakly percolated network in the solution. Thus, any suitable solvent can be selected to form a polymer-solvent combination existing in a percolated state which allows the liquid composition to be drawn into a fibre, the liquid composition may have a shear compliance of between around 10⁻³ to 10⁰ Pa⁻¹ (preferably between around 10⁻² to 10⁰ Pa⁻¹) at the measurement stress of 1 Pa at 25° C.

The liquid composition may have a total solid mass of less than 10% (w/v), preferably 5% (w/v), more preferably less than 3% (w/v), even more preferably less than 2.5% (w/v). For example, the total solid mass of the liquid composition can be between 0.1% to 5% (w/v), preferably between 0.5 to 3% (w/v), more preferably between 1 to 3% (w/v), most preferably between 2 to 2.5% (w/v). The increased fibre “wetness” (e.g., which can be exhibited when the total solid mass is less than 5% (w/v)) leads to more contributions from surface tension to form conformal contact (upon fibre drawing). While such a “wet” fibre can make it more difficult to draw a continuous fibre using conventional methods, such as in situ-spinning, the present methods and apparatus allow for fibres to be drawn from such solutions, thus achieving the improved functionality (e.g., specific fibre patterns, increased utility, such as conductive or pH-responsive devices) described herein.

The solvent may comprise water and/or an organic solvent. In some examples, the solvent comprises, or consists essentially of, water.

Preferably, the solvent comprises, or consists essentially of, an organic solvent. Organic solvent may include, but are not limited to alcohols, aromatic hydrocarbons, ketones, diacetone alcohol, dimethylformamide (DMF), n-methyl-2-pyrrolidone, butyrolactone, ethyl acetate, isopropyl acetate, propionic acid butyl ester, or mixtures of two or more thereof. Suitable alcohols include ethylene glycol monobutyl ether, ethylene glycol monomethyl ether, diethylene glycol monobutyl ether, diethylene glycol monomethyl ether, diethylene glycol monoethyl ether, hexanol, octanol, ethanol, methanol, 2-propanol, isopropanol, butanol, n-butanol, tert-butanol, ethylene glycol and diethylene glycol. Suitable aromatic hydrocarbons include benzene, toluene and xylene, ethylbenzene and cumene. Suitable ketones include acetone, methyl ethyl ketone, methyl isobutyl ketone and cyclohexanone. In some examples, the solvent may be selected from the group consisting of acetone, ethanol, methanol, 2-propanol, ethyl acetate, isopropyl acetate, methyl ethyl ketone, 1-butanol, and tert-butanol and mixtures of one or more thereof. In some examples, the organic solvent comprises dimethylformamide (DMF) and/or acetone. For example, the organic solvent may comprise DMF and acetone, preferably in a ratio of about 1.5:1 (v/v). In some examples, the solvent comprises water and/or acetone.

In some examples, the liquid composition may comprise PEO as the fibre-forming polymer and the solvent comprises, or consists essentially of, water. In other examples, the liquid composition may comprise PEO and HA as the fibre-forming polymer and the solvent comprises, or consists essentially of, water. In other examples, the liquid composition may comprise polystyrene as the fibre-forming polymer and the solvent comprises, or consists essentially of, DMF and/or acetone. Preferably, the solvent comprises DMF and acetone.

The functional component, as used herein, refers to a component suitable for use as a sensing element (e.g., including skin-interfaced bioelectric sensing and plant interface sensing). In other words, the functional component is capable of providing information resulting from sensing various properties from a location on a target. The weight ratio of the solid components of the fibre-forming polymer to the functional component may be about 1:1 to 5, preferably about 1:1 to 3, more preferably about 1:1.

In some examples, the fibre-forming polymer and the functional component may be the same component. In other words, in some examples, the fibre-forming polymer is the functional component. In such cases, the liquid composition of the present disclosure comprises a fibre-forming polymer and a solvent.

The functional component can comprise an electrically-conductive component and/or a responsive component such as colour-responsive component. For example, the functional component can be used to provide multi-modal sensing elements at the same location on the target. The functional component can be exchangeable or combinable, depending on the sensing function required.

In some examples, the functional component comprises, or consists essentially of an electrically-conductive component. A number of electrically-conductive components may be used in the composition of the present disclosure, suitable for forming an electrically-conductive drawn fibre. Typically, the electrically-conductive component is selected depending on its suitability as a bioelectric sensor. The electrically-conductive component can be selected from the group consisting of a conductive polymer, conductive carbon particles (optionally carbon nanotubes), metal nanoparticles (for example, silver nanoparticles), metal microparticles and mixtures of two or more thereof. In some examples, a suitable conductivity of about 104 to 106 S/m can be achieved.

In some examples, the electrically-conductive component comprises a conductive polymer. Preferably, the conductive polymer comprises one or more ionomer(s). The one or more ionomer(s) preferably comprise poly(3,4-ethylenedioxythiophene) and/or polystyrene sulfonate. Preferably, the electrically-conductive component comprises, or consists essentially of poly(3,4-ethylenedioxythiophene) polystyrene sulfonate (PEDOT:PSS). In some examples of the liquid composition, the fibre-forming polymer is PEO and the functional component is an electrically-conductive component. Preferably, the solvent comprises, or consists essentially of, water. The electrically-conductive component preferably comprises a conductive polymer. For example, the conductive polymer may be PEDOT:PSS. The weight ratio of the solid components of PEO to PEDOT:PSS in the composition is preferably about 1 to 5:1, optionally 1 to 3:1, optionally 1 to 2:1, optionally about 1:1. The liquid composition may further comprise hyaluronic acid and/or ethylene glycol. In some examples, the fibre-forming liquid composition comprises nanoparticles, such as silver nanoparticles (AgNP). The fibre-forming liquid composition may be prepared by mixing silver ink with the polymer matrix in a 1:1 volume ratio.

Where the fibre-forming liquid composition comprises nanoparticles, the method may include sintering the deposited fibre(s). For example, the deposited fibre(s) may be sintered at 100° C. for 15 minutes.

In some examples, the functional component comprises, or consists essentially of a responsive component such as a colour-responsive component. A number of colour-responsive components may be used in the composition of the present disclosure, suitable for forming a colour-responsive drawn fibre. Typically, a colour-responsive component is suitable as a colour-sensor e.g., by providing information from a location on a target based on colour responses (e.g. a targeted pH colour change). Thus, the pH indicator can be tailored and selected depending on its functional use. Preferably, the colour-responsive component comprises a pH indicator, also herein referred to as a “pH-responsive component” which provides a colour change based on its pH range. Any suitable pH indicators can be used in the composition for the present disclosure. For example, the pH indicator includes, but is not limited to, phenol red, universal pH indicator, nitrazine yellow, thymol blue, bromophenol blue, methyl orange, congo red indicator, bromocresol green, alizarin sulfonate sodium indicator, methyl red indicator, litmus, bromocresol purple, bromothymol blue indicator, neutral red, cresol red indicator, curcumin, phenolphthalein solution, malachite green etc. For example, the pH indicator can have a pH range of 5 to 9. Preferably, the pH indicator is phenol red.

In some examples, the liquid composition may comprise PEO as the fibre-forming polymer and a colour-responsive component as the functional component. Preferably, the colour-responsive component comprises a pH indicator. More preferably, the solvent comprises, or consists essentially of, water and/or acetone (most preferably water and acetone). The liquid composition may further comprise cellulose (e.g., cellulose nanofibers).

### EXAMPLES

### Example 1: Liquid Composition for Electrically-Conductive Fibres

An exemplary method of preparing a liquid composition (e.g., a fibre-forming liquid composition as described herein) is described below. The liquid composition for forming electrically-conductive fibres is prepared by mixing conducting PEDOT:PSS solution and viscoelastic PEO solution. The PEDOT:PSS solution is prepared by mixing 95% (v/v) of PEDOT:PSS and 5% (v/v) of ethylene glycol, and 1 drop (approximately 10 μL) of dodecylbenzenesulfonic acid (DBSA) is added to per 10 mL of the solution as a surfactant to prevent aggregation. The solution is then sonicated for around 20 min. The PEO solution is prepared by dissolving 2% (w/w) 8 M Da PEO and 0.5% (w/w) sodium hyaluronate in deionised (DI) water by mild stirring at room temperature (e.g., about 20.0° C.) for 48 hours. Before fibres can be drawn, the PEDOT:PSS solution and PEO solution are mixed together in 2:1 (v/v) ratio and stirred for 12 hour to form the liquid composition. The fibre-forming liquid composition can be stored in a fridge for use within two weeks.

### Example 2: Liquid Composition for Colour-Responsive Fibres

A liquid composition (e.g., a fibre-forming liquid composition as described herein) for forming colour-responsive drawn fibres is prepared by doping cellulose solution with colorimetric sensing dyes. 1.6% (w/v) 8 M Da PEO, 3% (w/v) cellulose nanofibres (3% (w/w) aqueous slurry) and 2% (w/v) phenol red are dissolved in acetone through stirring for 24 hours under room temperature (e.g., about 20.0° C.).

### Example 3: Method of Drawing a Fibre

An exemplary method of drawing a fibre is described below. A fibre-forming liquid composition prepared according to Example 1 is loaded into a 1 ml syringe and connected to a 22-gauge blunt-end stainless steel needle. Pressurised air, for example, at a pressure between 30 mbar and 50 mbar, is connected to the syringe to pump the fibre-forming liquid composition. In one example, approximately 30 mbar is used for the conducting PEDOT:PSS fibre-forming liquid composition 65. In another examples, approximately 50 mbar is used for the organic cellulose fibre-forming liquid composition. The syringe is placed above the arm 35 that constantly stretches the pendent droplet to initiate a thread jet, and the fibre jet subsequently lands and wraps onto the target object 1 in the centre of the deposition zone 45. The arm 35 is powered by a servo motor (e.g., a Parallax 6 V continuous servo) at one revolution per second. The radius of the rotation depends on the size of the target object, and can vary from 5 mm to 100 mm. The syringe and the rotational arm 35 form the fibre printing mechanism that could be coupled to the coupling 20 to allow in-situ adjustment of fibre printing angle and direction to adapt to the target object. It would be apparent that the speed of the servo motor can be adjusted to move the arm at different speeds depending on the application.

Throughout the description and claims of this specification, the words “comprise” and “contain” and variations of them mean “including but not limited to”, and they are not intended to (and do not) exclude other moieties, additives, components, integers or steps. Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, or groups described in conjunction with a particular aspect, embodiment or example of the invention are to be understood to be applicable to any other aspect, embodiment or example described herein unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The invention is not restricted to the details of any foregoing embodiments. The invention extends to any novel one, or any novel combination, of the features disclosed in this specification (including any accompanying claims, abstract and drawings), or to any novel one, or any novel combination, of the steps of any method or process so disclosed.

It will be appreciated by persons skilled in the art that the above embodiment(s) have been described by way of example only and not in any limitative sense, and that various alterations and modifications are possible without departing from the scope of the invention as defined by the appended claims. Various modifications to the detailed designs as described above are possible.

1. A method of depositing a fibre onto a freestanding object using a fibre deposition apparatus comprising a dispenser and a moveable arm defining a deposition zone, the method comprising:
positioning the fibre deposition apparatus such that the freestanding object is located within the deposition zone,
forming a droplet of a fibre-forming liquid composition at an outlet of the dispenser,
moving the arm from a first position in contact with the droplet to a second position away from the first position so as to draw the droplet into a drawn fibre, and
depositing the drawn fibre onto the freestanding object as the arm moves from the first position to the second position.

2. The method of claim 1, wherein the fibre is drawn in a wet state, for example a wet sol-gel state.

3. The method of claim 2, wherein the drawn fibre is deposited onto the freestanding object in a wet state, for example a wet sol-gel state.

4. The method of claim 3, wherein the drawn fibre is deposited onto the freestanding object in a Cassie-like state or in a Wenzel-like state.

5. The method of any preceding claim, wherein the arm comprises a tip that contacts the droplet, and wherein the arm is sized such that the tip is spaced from the freestanding object during movement of the arm relative to the freestanding object.

6. The method of any preceding claim, wherein the arm is rotated from the first position to the second position.

7. The method of claim 6, wherein the arm is rotated to orbit the freestanding object.

8. The method of claim 6 or claim 7, wherein the arm is rotated at a rate of less than around 100 revolutions per minute, RPM, for example about 90 RPM, for example about 80 RPM, for example about 70 RPM, for example between about 30 RPM and about 65 RPM, for example between about 45 RPM and about 65 RPM, for example about 60 RPM.

9. The method of any of claims 5 to 8, wherein the arm is rotated about a first axis to orbit the freestanding object, and wherein the method further comprises rotating the arm about a second axis different to the first axis.

10. The method of any preceding claim, further comprising translating the fibre deposition apparatus to generate relative movement between the arm and the freestanding object.

11. The method of claim 10, comprising translating the fibre deposition apparatus during movement of the arm between the first position and the second position.

12. The method of any preceding claim, wherein during depositing of the drawn fibre the freestanding object is stationary.

13. The method of any preceding claim, comprising drawing a plurality of fibres and depositing the plurality of fibres onto the freestanding object.

14. The method of claim 13, wherein a fibre density of the plurality of fibres deposited onto the freestanding object is between about 50 fibres per millimetre to about 5 fibres per centimetre.

15. The method of any preceding claim, comprising depositing the drawn fibre on a conductive component attached to the freestanding object.

16. The method of any preceding claim, comprising depositing the drawn fibre to bind a secondary object onto the freestanding object.

17. The method of claim 16, wherein the secondary object is an electronics component.

18. The method of any preceding claim comprising, depositing one or more additional layers onto the fibre, optionally wherein the one or more additional layers comprises a protective layer.

19. The method of claim 18 wherein the fibre is a first fibre and depositing the protective layer onto the first fibre comprises depositing a second fibre onto the first fibre using the fibre deposition apparatus, optionally wherein the second fibre comprises cellulose.

20. One or more fibres deposited according to the method of any preceding claim.

21. A yarn of one or more fibres, formed by the method according to any one of claims 1 to 14, wherein the method involves depositing the one or more fibres onto a support and removing the one or more fibres from the support to form the yarn.

22. A fibre deposition apparatus for depositing fibres onto a freestanding object, the fibre deposition apparatus comprising:
a dispenser having an outlet for forming a droplet of a fibre-forming liquid composition; and
an arm arranged to move relative to the dispenser from a first position in which the arm contacts the droplet of fibre-forming liquid composition to a second position away from the first position so as to draw the droplet into a drawn fibre,
wherein the arm is arranged to deposit the drawn fibre onto the freestanding object located within a deposition zone defined by the movement of the arm.

23. The fibre deposition apparatus of claim 22, wherein the arm is configured to draw the fibre in a wet state, for example a wet sol-gel state.

24. The fibre deposition apparatus of claim 23, wherein the fibre deposition apparatus is configured to deposit the drawn fibre onto the freestanding object in a wet state, for example a wet sol-gel state.

25. The fibre deposition apparatus of any of claims 22 to 24, wherein the arm comprises a tip that contacts the droplet, and wherein the arm is sized such that the tip is spaced from the freestanding object during movement of the arm relative to the freestanding object.

26. The fibre deposition apparatus of any of claims 22 to 25, wherein the arm is rotatable from the first position to the second position.

27. The fibre deposition apparatus of claim 26, wherein the arm is rotatable to orbit the object.

28. The fibre deposition apparatus of claim 26 or claim 27, wherein the arm is rotatable at a rate of less than about 100 revolutions per minute, RPM, for example less than about 90 RPM, for example less than about 80 RPM, for example less than about 70 RPM, for example between about 30 RPM and about 65 RPM, for example between about 45 RPM and about 65 RPM, for example about 60 RPM.

29. The fibre deposition apparatus of any of claims 26 to 28, wherein the arm is rotatable about a first axis to orbit the object, and wherein the arm is further rotatable about a second axis different to the first axis.

30. The fibre deposition apparatus of any of claims 22 to 29 wherein the apparatus includes a multi-axis robotic arm.

31. The fibre deposition apparatus of any of claims 22 to 30, further comprising an actuator operable to translate the arm to generate relative movement between the arm and the object.

32. The fibre deposition apparatus of claim 31, wherein the actuator is operable to translate the arm during movement of the arm between the first position and the second position.

33. The fibre deposition apparatus of claim 31 or claim 32, wherein the actuator is operable to translate the arm such that a fibre density of the plurality of fibres deposited onto the freestanding object is between about 50 fibres per millimetre to about 5 fibres per centimetre.

34. The fibre deposition apparatus of any of claims 22 to 33, further comprising a support frame, wherein the arm is mounted to the support frame, and further comprising a coupling arranged to mount the support frame to a stage, wherein the coupling is arranged to provide at least one degree of freedom of movement between the support frame and the stage so as to allow a user to selectively position the support frame relative to the stage.

35. The fibre deposition apparatus of claim 34, wherein the coupling is lockable.

36. A liquid composition for forming a drawn fibre, comprising:
a fibre-forming polymer;
a solvent; and
a functional component.

37. The liquid composition of claim 36, wherein the liquid composition has a shear compliance of between 10⁻² to 100 Pa⁻¹.

38. The liquid composition according to claim 36 or claim 37, wherein the fibre-forming polymer comprises one or more polymers selected from the group consisting of polyethers, polycarbonates, polyamides, polyimides, polyolefins, halogenated polymers, aromatic polymers, ketone polymers, methacrylate polymers, polyesters, cellulose derivatives, polypeptides, polysulfones, polyurethanes, polyureas, glycosaminoglycans, polypeptides, polysaccharides, polyphenols, proteoglycans and polynucleotides.

39. The liquid composition according to any of claims 36 to 38, wherein the fibre-forming polymer comprises a polyether, polysaccharide, and/or an aromatic polymer, optionally wherein the fibre-forming polymer comprises polyethylene oxide (PEO), cellulose or its derivatives and/or polystyrene.

40. The liquid composition according to any of claims 36 to 39, wherein the fibre-forming polymer comprises a polyether and/or a glycosaminoglycan, optionally wherein the fibre-forming polymer comprises polyethylene oxide (PEO) and/or hyaluronic acid (HA).

41. The liquid composition according to any of claims 36 to 40, wherein the solvent comprises water and/or an organic solvent.

42. The liquid composition according to claim 41, wherein the organic solvent comprises dimethylformamide (DMF) and/or acetone.

43. The liquid composition according to any of claims 36 to 42, wherein the functional component comprises an electrically-conductive component and/or a colour-responsive component.

44. The liquid composition according to claim 43, wherein the electrically-conductive component is selected from the group consisting of a conductive polymer, conductive carbon particles (optionally carbon nanotubes), metal nanoparticles, metal microparticles and mixtures of two or more thereof.

45. The liquid composition according to claim 36, wherein:
the fibre-forming polymer comprises PEO; and
the functional component is an electrically-conductive component comprising a conductive polymer, optionally wherein the fibre-forming polymer comprises HA.

46. The liquid composition according to claim 44 or claim 45, wherein the conductive polymer comprises one or more ionomer(s).

47. The liquid composition according to claim 44 or claim 45, wherein the conductive polymer is poly(3,4-ethylenedioxythiophene) polystyrene sulfonate (PEDOT:PSS).

48. The liquid composition according to claim 47, wherein the weight ratio of PEO to PEDOT:PSS is about 1:1 to 5, optionally about 1:1 to 3, optionally about 1:1.

49. The liquid composition according to any of claims 36 to 48, wherein the solvent comprises water.

50. The liquid composition according to claim 36, wherein:
the fibre-forming polymer comprises polystyrene; and
the solvent comprises DMF and/or acetone.

51. The liquid composition according to claim 36, wherein:
the fibre-forming polymer comprises PEO; and
the functional component is a colour-responsive component.

52. The liquid composition according to claim 51, wherein the colour-responsive component comprises a pH indicator.

53. The liquid composition of any of claims 36 to 52, wherein the liquid composition has a total solid mass of less than 5% (w/v), optionally less than 3% (w/V).

54. The liquid composition of any one of claims 36 to 53, wherein the liquid composition is characterised by a ratio of the elastic storage modulus to the surface tension of between: 0.5 and 3.5; or 1 and 3; or 1.5 and 2.5.

55. The method according to claims 1 to 19, wherein the fibre-forming liquid composition is a liquid composition according to any of claims 36 to 54.

56. The fibre deposition apparatus according to claims 22 to 35, wherein the fibre-forming liquid composition is a liquid composition according to any of claims 36 to 54.

57. A sensor comprising a plurality of fibres drawn from a liquid composition and deposited onto a freestanding object.

58. The sensor according to claim 57, wherein the fibre-forming liquid composition is a liquid composition according to any of claims 36 to 54.

59. The sensor according to claim 57 or claim 58, wherein the sensor further comprises a conductive element and/or an electronics component located between the freestanding object and the plurality of fibres, optionally wherein the electronics component comprises an indicator.

60. The sensor according to any one of claims 57 to 59, wherein the sensor is operable to detect one or more of:
electrocardiography, ECG, data,
electromyography, EMG, data,
a pH of the freestanding object or the environment surrounding the freestanding object,
a concentration or presence of a target species of the freestanding object or the environment surrounding the freestanding object,
movement or transformation of the freestanding object,
temperature of the freestanding object,
pressure applied to the freestanding object, and/or
a strain of the freestanding object.

61. A device for heat generation, refrigeration and/or energy harvesting comprising a plurality of fibres drawn from a liquid composition and deposited onto a freestanding object, optionally wherein the plurality of fibres comprise a thermoelectric element.

62. An object comprising a plurality of fibres deposited thereon, wherein the plurality of fibres form an open array of fibres.

63. The object according to claim 63, wherein the plurality of fibres are configured as a regular array, optionally wherein the regular array is in the form of evenly spaced parallel lines, a square matrix, a fanning array, or a parallelogram array.

64. The object according to claim 63, wherein the plurality of fibres are configured as an irregular array, optionally wherein the irregular array is in the form of a programmable trace.

65. The object according to any one of claims 62 to 64, wherein the object comprises a textile and the plurality of fibres are deposited onto the textile, optionally wherein the textile is a yarn or natural fibres or human hair.

66. An object comprising a fibre deposited thereon, wherein the fibre is morphed to the surface topography such that the fibre is in contact with the surface topography at over 50% of its length when observed at a microscopic scale.

67. An object according to claim 66, wherein the fibre does not comprise a volatile solvent.

68. An object according to claim 66 or claim 67, wherein the fibre is an electrically-conductive fibre.

69. An object according to any one of claims 66 to 68, wherein the object comprises organic matter and wherein the fibre is deposited on the organic matter, optionally wherein the organic matter is skin or plant matter.

70. An object comprising a fibre deposited thereon, wherein the fibres are characterised by a maximum peeling force per unit fibre width of over 15 N, when measured according to a 90-degree peeling test.
